SEQUENCE LISTING (30 sequences)

SEQ ID NO: 1 - protein, 8 aa, Artificial Sequence
  Synthetic Peptide
MAVPCVVM

SEQ ID NO: 2 - protein, 8 aa, Artificial Sequence
  Synthetic Peptide
MAVPCVVM

SEQ ID NO: 3 - protein, 10 aa, Artificial Sequence
  Synthetic Peptide
CQDSETRTFY

SEQ ID NO: 4 - protein, 5 aa, Artificial Sequence
  Synthetic Peptide
CRANK

SEQ ID NO: 5 - protein, 5 aa, Artificial Sequence
  Synthetic Peptide
LYRAX

SEQ ID NO: 6 - protein, 10 aa, Artificial Sequence
  Synthetic Peptide
LYRAXCRANK

SEQ ID NO: 7 - protein, 4 aa, Artificial Sequence
  Synthetic Peptide
SXLT

SEQ ID NO: 8 - protein, 5 aa, Artificial Sequence
  Synthetic Peptide
LAXVT

SEQ ID NO: 9 - protein, 12 aa, Artificial Sequence
  Synthetic Peptide
GNXETSNTSSPS

SEQ ID NO: 10 - protein, 5 aa, Artificial Sequence
  Synthetic Peptide
XRANK

SEQ ID NO: 11 - protein, 10 aa, Artificial Sequence
  Synthetic Peptide
LYRAXXRANK

SEQ ID NO: 12 - protein, 42 aa, Artificial Sequence [sequence omitted]

SEQ ID NO: 13 - protein, 6 aa, Artificial Sequence
  Synthetic Peptide
DAEFRH

SEQ ID NO: 14 - protein, 13 aa, Artificial Sequence
  Synthetic Peptide
NSGYEVHHQKLVF

SEQ ID NO: 15 - protein, 7 aa, Artificial Sequence
  Synthetic Peptide
XAEDVGS

SEQ ID NO: 16 - protein, 16 aa, Artificial Sequence
  Synthetic Peptide
NKGAIIGLMVGGVVIA

SEQ ID NO: 17 - protein, 6 aa, Artificial Sequence
  Synthetic Peptide
DAEFRH

SEQ ID NO: 18 - protein, 13 aa, Artificial Sequence
  Synthetic Peptide
XSGYEVHHQKLVF

SEQ ID NO: 19 - protein, 19 aa, Artificial Sequence
  Synthetic Peptide
DAEFRHXSGYEVHHQKLVF

SEQ ID NO: 20 - protein, 19 aa, Artificial Sequence
  Synthetic Peptide
DAEFRHDSGYEVHHQKLVF

SEQ ID NO: 21 - protein, 26 aa, Artificial Sequence
  Synthetic Peptide
DAEFRHDSGYEVHHQKLVFFAEDVGS

SEQ ID NO: 22 - protein, 26 aa, Artificial Sequence
  Synthetic Peptide
DAEFRHDSGYEVHHQKLVFFAEDVGS

SEQ ID NO: 23 - protein, 16 aa, Artificial Sequence
  Synthetic Peptide
XKGAIIGLMVGGVVIA

SEQ ID NO: 24 - protein, 42 aa, Artificial Sequence
  Synthetic Peptide
DAEFRHDSGYEVHHQKLVFFAEDVGSXKGAIIGLMVGGVVIA

SEQ ID NO: 25 - protein, 42 aa, Artificial Sequence [sequence omitted]

SEQ ID NO: 26 - protein, 10 aa, Artificial Sequence
  Synthetic Peptide
CQDSETRTFY

SEQ ID NO: 27 - protein, 10 aa, Artificial Sequence
  Synthetic Peptide
CQDSETRTFY

SEQ ID NO: 28 - protein, 5 aa, Artificial Sequence
  Synthetic Peptide
XRANK

SEQ ID NO: 29 - protein, 5 aa, Artificial Sequence
  Synthetic Peptide
LYRAM

SEQ ID NO: 30 - protein, 10 aa, Artificial Sequence
  Synthetic Peptide
LYRAMXRANK

(19) United States
(12) Patent Application Publication
(10) Pub. No.: US 2010/0222546 A1
(43) Pub. Date: Sep. 2, 2010

(54) METHODS FOR THE PREPARATION OF FUNCTIONALIZED PEPTIDES, PROTEINS AND CARBOHYDRATES AND THEIR CONJUGATES

(76) Inventors: David Crich, Grosse Point Park, MI (US); Songpu Guo, Chicago, IL (US); Fan Yang, Detroit, MI (US); Kasinath Sana, Detroit, MI (US)

Correspondence Address: Olson & Cepuritis, LTD., 20 NORTH WACKER DRIVE, 36TH FLOOR, CHICAGO, IL, 60606, US

(21) Appl. No.: 12/312,766

(22) PCT Filed: Nov. 28, 2007
(86) PCT No.: PCT/US07/24456

§ 371 Date: May 26, 2009

Related U.S. Application Data
(60) Provisional application No. 60/861,380, filed on Nov. 28, 2006.

(51) Int. Cl. C07K 14/00; C07K 7/06; C07K 7/08; C07K 9/00

(52) U.S. Cl. 530/322; 530/328; 530/330; 530/327; 530/324; 530/326

(57) ABSTRACT

The present invention relates to methods for ligation or derivatization of peptides, amino acids, and carbohydrates utilizing a chalcogen-based reactant, a peptide reactant, an amino acid reactant, a chalcogen-containing peptide reactant, a chalcogen-containing amino acid reactant, or a combination of two or more of the foregoing reactants substantially as described herein.

### CROSS-REFERENCE TO RELATED APPLICATIONS

This application claims the benefit of U.S. Provisional Application Ser. No. 60/861,380, filed on Nov. 28, 2006, which is incorporated herein by reference.

### STATEMENT OF GOVERNMENTAL INTEREST

This research was supported by Grant Nos. GM62160 and GM080384 from the National Institutes of Health, Bethesda, Md. The United States government has certain rights to this invention.

### FIELD OF THE INVENTION

This invention generally relates to methods for ligating complex organic compounds such as peptides, amino acids, and carbohydrates with chalcogen-containing reagents.

### INTRODUCTION AND BACKGROUND

The ability to prepare and modify bioconjugates of all kinds under mild, and even physiologically relevant, conditions is critical to the developing fields of glycomics¹⁻⁶ and proteomics⁷⁻¹⁰ and is, thus, germane to the study of human disease states.

The present invention relates to the development of facile, efficient ligation methods for the assembly and modification of diversely functionalized peptides and oligosaccharides and their conjugates. The methodologies are operationally simple and suitable for a range of biomedical research.

The invention focuses on three main reaction types: the formation of permanent linkages to cysteine; the development of new and improved methodology for the formation N-glycosylated asparagine derivatives; and a novel extension of the concept of native chemical ligation to the formation of peptidic bonds to phenylalanine, tyrosine, tryptophan, aspartic acid and asparagine.

These new methods for the formation of permanent linkages to cysteine residues specifically avoid the use of electrophilic agents, with their selectivity problems, and focus on the classical disulfide ligation and on methods for rendering such ligation permanent by the excision of a single atom of sulfur (or selenium) to form a thioether linkage in place of a disulfide (or selenosulfide).

With respect to the formation of N-glycosyl asparagine residues, the methods avoid existing methods of amide glycosylation, and of amide bond formation to glycosyl amines, with their well-known limitations. The chemistry of electron-deficient sulfonamides and of native chemical ligation is adapted to the formation of these challenging, critically important linkage points in peptidoglycans.

One objective in the native chemical ligation field is to diversify this extremely important methodology by permitting peptidic bonds to be formed to the nitrogen of phenylalanine, tyrosine, tryptophan, aspartic acid and asparagine. The precursors for this chemistry are assembled in a strict minimum of steps, from readily accessible materials, their incorporation into peptide sequences is compatible with existing peptide synthesis techniques, and the removal of any handles post-ligation is limited to a maximum of one or two simple steps.

The present methods are useful in the synthesis of target functionalized peptides, glycoconjugates and peptides, which are of wide ranging importance in the manufacture of drugs and drug candidates.

### SUMMARY OF THE INVENTION

As used herein, “chemical ligation” is defined broadly as any reaction capable of joining together through covalent bond formation two or more chemical entities under mild conditions, preferably approaching physiological conditions, so as to facilitate the preparation and study of complex biologically active substances. Chemical entities include the traditional types of biopolymers (oligosaccharides, peptides and proteins, oligonucleotides, lipids), and their conjugates and neoconjugates, and any unit that is desirable to attach to them, such as polyethylene glycol chains, fluorous groups, biotin, spin labels, fluorescent markers, etc. The present invention provides improved methodologies for chemical ligation, which are broadly applicable, straightforward to conduct by the non-specialist, and of a robust nature. The present methods relate to peptide and protein modification with particular emphasis on lipidation, glycosylation,^{11, 12} and the preparation of neoglycoconjugates^{13, 14} and neoconjugates in general.

Term “protecting group” and grammatical variations thereof, as used herein, refers to organic moieties attached to functional groups (e.g., on an oxygen, nitrogen, or sulfur atom in the functional group), typically used during preparation of complex molecules such as amino acids, peptides, monosaccharides, polysaccharides, and the like, which can be selectively removed to unmask the functional group of the complex molecule when a synthetic procedure is complete. Protecting groups for various functional groups such as amines, thiols, alcohols, carboxylic acids, and the like are very well known to those of ordinary skill in the organic chemical arts. A compendium of many of the most common protecting groups used in the synthesis of complex compounds has been assembled by Theodora W. Greene and Peter G. M. Wuts, in a book entitled “Protective Groups in Organic Synthesis”, the third edition of which was published by John Wiley & Sons, New York in 1999; the relevant portions of which are incorporated herein by reference.

The present invention relates to methods for ligation or derivatization of peptides. amino acids, and carbohydrates utilizing a chalcogen-based reactant, a peptide reactant, an amino acid reactant, a chalcogen-containing peptide reactant, a chalcogen-containing amino acid reactant, or a combination of two or more of the foregoing reactants substantially as described herein.

In one aspect, the method comprises reacting an amino disulfide compound with a thioester reactant in the presence of a thiol such as 2-mercaptoethylsulfonate salt to form a peptide between the amino group and the thioester, and concomitantly reductively cleaving the disulfide bond of the disulfide group to form a free SH group on the peptide. Optionally, the free SH group is reductively cleaved from the peptide and replaced by a hydrogen.

In yet another aspect, the present invention provides a ligation method comprising contacting a nitroarylsulfonamide with a thiocarboxylic acid in the presence of a base, thereby forming an amide bond between the nitrogen of the sulfonamide and the carbonyl of the thiocarboxylic acid. Alternatively, a thioester-containing compound can be contacted with a thioaspartic acid compound including a free amino group (e.g., an N-terminal thioaspartic acid residue of a peptide), thereby forming a peptide bond between the free amino group and the carbonyl moiety of the thioester group.

A preferred method aspect of the present invention provides for ligation or derivatization of a peptide. The method comprises reacting sulfonamide (I) with peptide (II) to form ligated peptide (III):

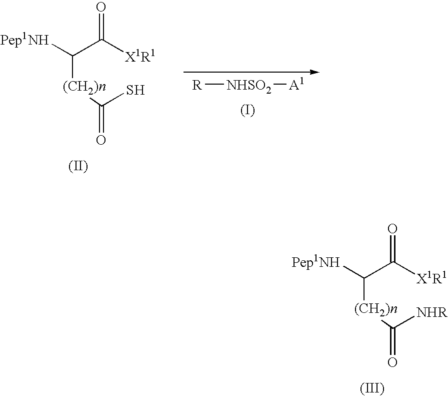

wherein R is an amino acid, a peptide, a monosaccharide, or a polysaccharide, optionally including one or more protecting groups on a nitrogen, oxygen, or sulfur substitutent thereof; X¹ is O or NH; R¹ is an alkyl group, an alkenyl group, an aryl group, an alkyl-substituted aryl group, an aryl-substituted alkyl group, an amino acid, or a peptide, optionally including one or more protecting groups on a nitrogen, oxygen, or sulfur substitutent thereof; A¹ is an electron deficient alkyl, aryl, or heteroaryl group; Pep¹ is an amino acid or a peptide, which optionally includes one or more protecting groups on a nitrogen, oxygen, or sulfur substitutent thereof; and n is 1 or 2.

Another preferred method aspect of the present invention provides for glycosylation of a peptide. The method comprises reacting a N-glycosyl-o-nitrobenzylamino-disulfide compound (Glyc¹-NHZ) with peptidyl thioester (IV) in the presence of a thiol, and subsequent photolysis to afford glycosylated peptide (V):

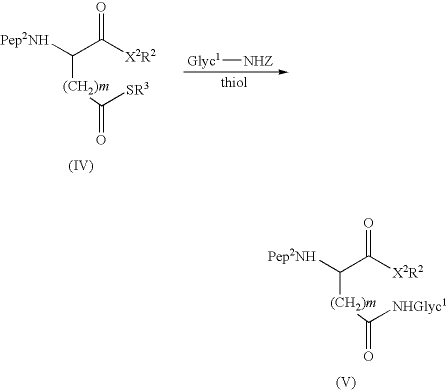

wherein X² is O or NH; R² is an alkyl group, an alkenyl group, an aryl group, an alkyl-substituted aryl group, an aryl-substituted alkyl group, an amino acid, or a peptide, optionally including one or more protecting groups on a nitrogen, oxygen, or sulfur substitutent thereof; R³ is an alkyl group, an alkenyl group, an aryl group, an alkyl-substituted aryl group, or an aryl-substituted alkyl group; Pep² is an amino acid or a peptide, which optionally includes one or more protecting groups on a nitrogen, oxygen, or sulfur substitutent thereof; Glyc¹ is a monosaccharide or a polysaccharide, optionally including one or more protecting groups on a nitrogen, oxygen, or sulfur substitutent thereof; Z is an o-nitrobenzyl-disulfide moiety; and m is 1 or 2.

Yet another method aspect of the invention provides for native chemical ligation of a peptide at a phenylalanine, tyrosine or tryptophan residue, and comprises reacting peptidyl thioester (IV) with aminodisulfide (VI) in the presence of a thiol to afford mercapto peptide (VII); and optionally reducing (VII) to afford peptide (VIII):

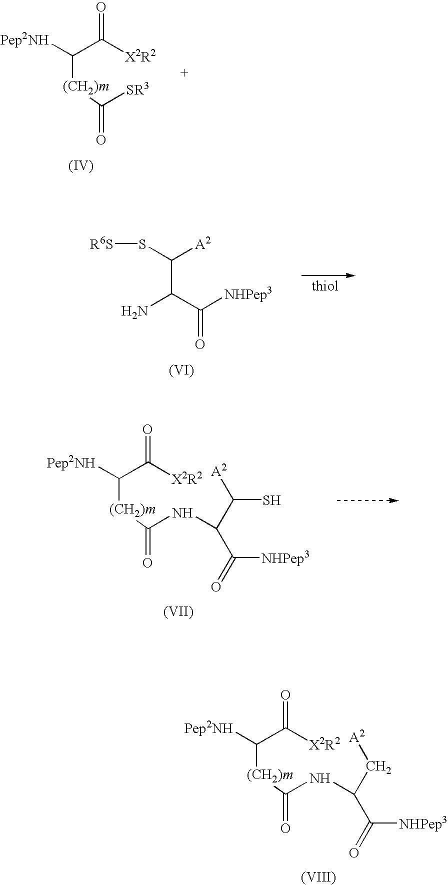

wherein X² is O or NH; R² is an alkyl group, an alkenyl group, an aryl group, an alkyl-substituted aryl group, an aryl-substituted alkyl group, an amino acid, or a peptide, optionally including one or more protecting groups on a nitrogen, oxygen, or sulfur substitutent thereof; R³ and R⁶ are each independently an alkyl group, an alkenyl group, an aryl group, an alkyl-substituted aryl group, or an aryl-substituted alkyl group; Pep² and Pep³ are each independently an amino acid or a peptide, which optionally includes one or more protecting groups on a nitrogen, oxygen, or sulfur substitutent thereof; A² is phenyl, 4-hydroxyphenyl, or 3-indolyl; and m is 1 or 2.

### DETAILED DESCRIPTION OF THE INVENTION

The ability to functionalize peptides and proteins is very widely recognized to be a critical tool in the study of a very broad spectrum of disease states. Reasons vary from the access that such functionalization provides to larger scale quantities of materials that are otherwise only available as minute amounts of inhomogeneous isolates, to the need to tag molecules with fluorescent or other markers so as to monitor their function in living cells. The importance of chemistry to this problem is brought home by the significant number of recent reviews in chemistry related journals,¹⁵⁻²⁰ and is highlighted by the number of recent publications dealing with methods for peptide and protein glycosylation and lipidation, whether in the native form or as neoconjugates.²¹⁻³⁵ The following brief overview relates to a select set of ligation reactions which are relevant to the chemistry discussed herein.

One of the most popular ligation methods is the so-called Staudinger ligation developed by the Bertozzi group.³⁶⁻⁴⁰ This method has enjoyed enormous success in the chemical biology field in recent years.^{15, 41, 42} In this reaction, using the principles of the classical Staudinger reaction between an azide and a phosphine,⁴³ an o-diphenylphosphinobenzoate ester captures an alkyl azide in the form of an iminophosphorane.⁴⁴ The nitrogen is then transferred intramolecularly to the ester with formation of an amide linkage, thereby achieving the ligation of the groups R and R′ carried by the original phosphine and azide (Scheme 1).

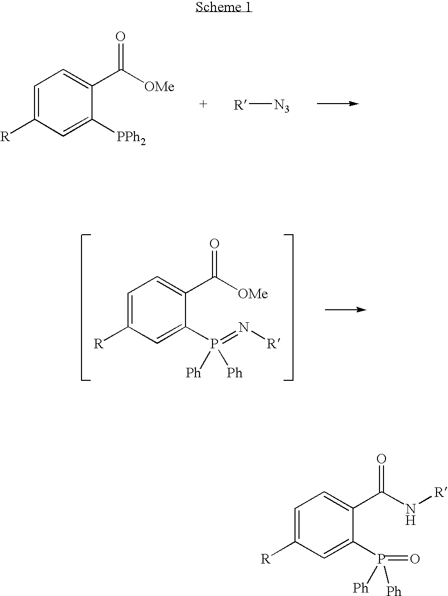

In the so-called traceless Staudinger ligation the R group to be ligated is carried in the form of an ester ortho to the phosphine, resulting overall in the formation of a simple amide bond and the expulsion of the phosphine oxide byproduct (Scheme 2).⁴⁵

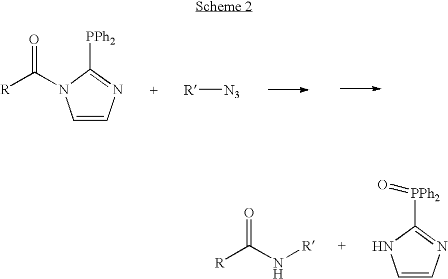

A further ligation process that makes use of alkyl (and aryl) azides is their reaction with thioacids to give amides. This reaction, which is applicable to carbohydrate-based azides as we and others have demonstrated,^{46, 47} has been extensively studied recently by the Williams^{48, 49} and Liskamp groups (Scheme 3).⁵⁰ A modification of this reaction employing selenocarboxylates has been developed,⁵¹ and it has been shown that dithioacids afford thioamides on reaction with azides.⁵² Unfortunately, the temperatures required for the use of alkyl azides (˜60° C.) appear to preclude the use of this reaction with sensitive biopolymers.

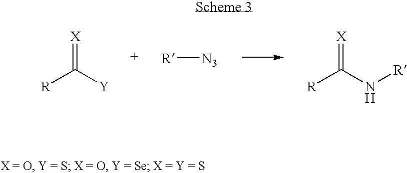

A form of native chemical ligation was introduced by Kent and co-workers, which has contributed enormously to the chemical synthesis of peptides and proteins,⁵³⁻⁶⁰ and even peptide nucleic acids.⁶¹ The key steps in this reaction are the facile transesterification of a peptide-based C-terminal thiol ester with the thiol of an N-terminal cysteine in a second peptide to give a new thiol ester linking the two peptides, and the intramolecular S to N transfer giving the native peptide bond (Scheme 4). The mechanism, and especially the use of auxiliary thiols, has been studied in some detail, and the method has recently been conducted on soluble polymeric supports.^{62, 63}

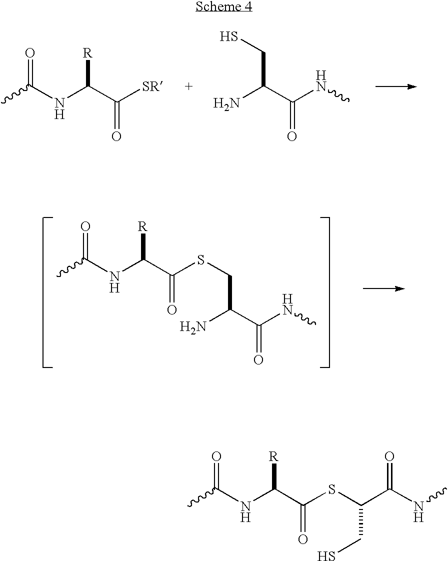

Several modifications have been designed to circumvent the mechanistic requirement for the N-terminal cysteine residue in this widely applied chemistry. The simplest of these is desulfurization after ligation which leads to the conversion of the cysteine moiety into an alanine group,⁶⁴ and, in combination with the use of selenocysteine with the more nucleophilic selenol group and the more easily cleaved Se—C bond, this should be a powerful method.⁶⁵ More typically, however, auxiliaries have been designed which incorporate a thiol group positioned so as to be cleaved without trace after the ligation reaction. Initially, an N-terminal N-(2-mercaptoethoxy) group was used to deliver the amine, with subsequent reductive cleavage of the N—O bond,⁶⁶ but this has been replaced by an approach, developed by several groups, involving the use of an α-benzyl-3-mercaptoethyl amine which is readily removed after ligation thanks to the enhanced lability of the benzylic C—N bond. Examples of this type include the Kent auxiliary (Scheme 5),⁶⁷ and improved but closely related systems developed by Clive and Botti.^{68, 69} Systems based on similar design principles have also been described by Dawson, Tam, and others.⁷⁰⁻⁷⁴ An interesting extension of the method to the synthesis of N-(N-acetylglucosaminyl)asparagine-based peptides described recently makes use of an N-(N-2-mercaptoacetylglucosaminyl)asparagine as the N-terminal amino acid with capture of the thiol ester by the mercaptoacetyl group on the carbohydrate and transfer of the S- to N-linkage through an eleven-membered cyclic intermediate.⁷⁵ The final stage of this peptidoglycan synthesis is desulfurization of the mercapto-acetamide unit to the requisite acetamide.

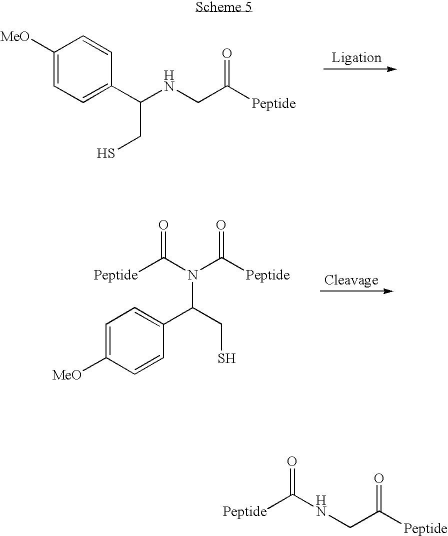

Combining the concept of rapid thiol ester exchange central to native chemical ligation with that of the traceless Staudinger reaction developed by Bertozzi (Scheme 2),⁴⁵ the Raines and Kiessling groups developed diphenylphosphinylmethanethiol,⁷⁶ and employed it in the synthesis of diverse bioconjugates. When the azide employed is an α-azido acid, or more specifically a peptide derivative thereof, the method constitutes a formal peptide synthesis.^{76, 77} One of the more novel and relevant applications of this chemistry has been in the synthesis of N-glycosyl asparagine residues by the reaction of a glycosyl azide with an aspartic acid side chain thiol ester (Scheme 6).^{78, 79} However, this method appears to suffer from equilibration of the anomeric stereochemistry at the level of the intermediate iminophosphorane, which results in the formation of anomeric mixtures. Most recently, it has been shown that in the presence of tributylphosphine glycosyl azides react with the standard aspartic acid side chain, activated in the form of a hydroxybenzotriazole ester, to give the N-glycosylated asparagines derivatives directly—that is by intermolecular transfer of the iminophosphorane with the activated ester.⁸⁰

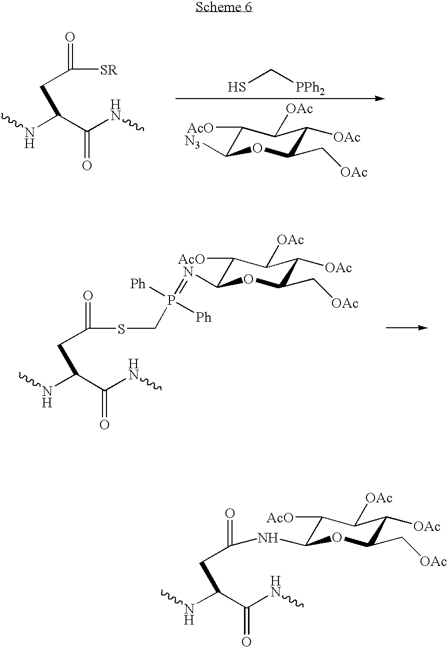

A more classical ligation reaction with which most chemists and biochemists are familiar is the disulfide ligation in which a thiol is first reacted with dipyridyl disulfide, or an activated form thereof, to give an alkyl pyridyl disulfide. This mixed disulfide is then allowed to react with a second thiol leading overall to the formation of a mixed dialkyl disulfide (Scheme 7).⁸¹⁻⁸³

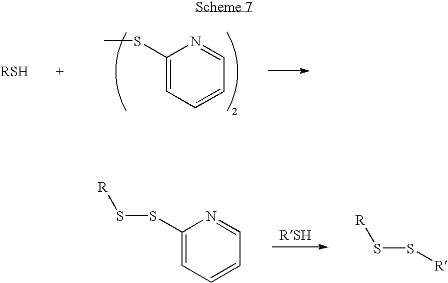

This reaction is in extremely widespread use for the formation of bioconjugates, especially for the derivatization of cysteine in peptides and proteins.^{84, 85} Noteworthy in the context of this proposal is the application to the glycosylation of peptides by Boons (Scheme 8, X=S-5-nitropyridyl),⁸⁶ and the more recent modifications developed by Davis employing phenylthiosulfonates, or selenosulfides, rather than a pyridyl sulfide to deliver the thiol (Scheme 8, X=SePh), and successfully employed in the glycosylation of proteins.^{87, 88}

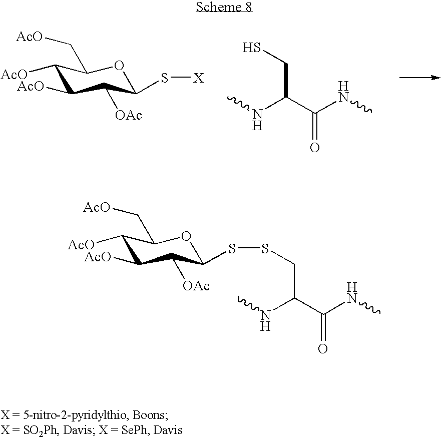

The mildness of the disulfide ligation and its established chemoselectivity for the cysteine thiol in the presence of all the proteinogenic amino acids is in stark contrast to the various other methods for cysteine functionalization, most of which involve the capture of the cysteine thiol by electrophilic species, and which consequently have obvious potential chemoselectivity issues in many circumstances.^{84, 85, 89} The practicality of the disulfide ligation, with its direct applicability to cysteine-containing peptides, also contrasts with the various ingenious indirect methods that have been developed for the preparation of S-functionalized cysteine derivatives,¹² including, for example, the Michael addition of thiols to dehydroalanine units,⁹⁰ the alkylation of thiolates with peptide-based β-haloalanine units,^{24, 29, 30} and other electrophiles,^{31, 91} the opening of peptide-based aziridines by thiolates,^{23, 92} and the synthesis of peptides with previously functionalized cysteine building blocks,^{32, 93, 94} each of which requires the synthesis of modified peptides. The many advantages of the disulfide ligation are offset, however, by its impermanence which results from the lability of the disulfide bond in the presence of thiols and other reducing agents. Consideration of the practical advantages of the disulfide ligation, and the disadvantages of its impermanence, lead to the development of new methods for cysteine functionalization as described herein.

### Initial Results

### Cysteine Functionalization

In one embodiment, the present invention relates to a first generation method for the ligation of alkyl groups to thiols, especially cysteine residues. In accordance with reactions involving simple alkyl halides,⁹⁵⁻⁹⁷ primary allylic halides are reacted with potassium selenosulfate to form a series of Se-allylic selenosulfate, or Se-Bunte salts (Scheme 9). For the most part, these Se-Bunte salts are orange crystalline solids that can be readily manipulated and stored in the refrigerator for weeks without extensive decomposition. Again with regard to simple Se-alkyl Se-Bunte salts,⁹⁸ reaction of these Se-allylic Bunte salts with simple thiols at room temperature rapidly yields Se-allyl S-alkyl selenosulfides (Scheme 9).

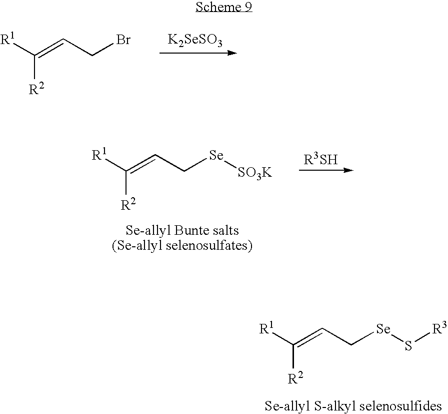

These Se-allyl S-alkyl selenosulfides can be readily observed on thin layer chromatography (TLC) and even by NMR at room temperature. Over a period of several hours at room temperature they undergo a deselenative 2,3-sigmatropic rearrangement to give regiochemically inverted allyl alkyl sulfides (Scheme 9). The proposed intermediate selenosulfoxides are not observed by NMR suggesting that the initial equilibrium heavily favors the Se-allyl S-alkyl selenosulfides. Depending on the nature of the R¹ and R² groups at the distal end of the alkene the transient selenosulfoxides undergo loss of selenium either spontaneously (R¹=R²=H) or on addition of a phosphine (R¹, R²=alkyl) (Scheme 10). The mechanism of the “spontaneous” loss of selenium has yet to be determined, and may involve reaction with the solvent or the reaction of two or more molecules of selenosulfoxide with each other. We view the differing requirements of the final deselenative step as demonstrative of a shift in the selenosulfide/selenosulfoxide equilibrium according to the nature of the substituents. Thus, when R¹ and R² are hydrogen a primary carbon sulfur bond is formed at the expense of a primary carbon selenium bond and the selenosulfoxide has sufficient lifetime to undergo the apparently spontaneous loss of selenium. On the other hand, when R¹ and R² are alkyl a weaker tertiary carbon sulfur bond is formed at the expense of a primary carbon selenium bond and a monosubstituted alkene is formed from a trisubstituted alkene—overall a significantly less favorable process—which reduces the lifetime of the selenosulfoxide, and necessitates the addition of phosphine to drive the equilibrium in the forward direction. The pattern of dependency of the deselenative rearrangement on the nature of the R¹ and R² groups mirrors that reported originally by Baldwin for his diallyl disulfide rearrangements.⁹⁹

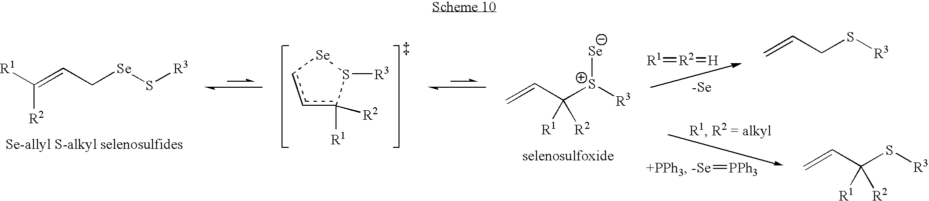

Using this chemistry we have succeeded in the allylation of a number of thiols at room temperature in methanol solution, as we communicated recently in the Journal of the American Chemical Society,¹⁰⁰ and as highlighted in the Feb. 27, 2006 issue of Chemical and Engineering News. Illustrative examples of the new ligation method are set out in Scheme 11. Noteworthy among these examples is the formation of the allylically transposed S-nerolidyl tripeptide starting from the Se-farnesyl Bunte salt, which nicely confirms the 2,3-sigmatropic nature of the rearrangement, and the formation of the S-allyl thioglycoside which took place with complete retention of the anomeric stereochemistry. It should also be noted that, when the reactions were run in methanol D₄, no incorporation of deuterium into the peptide α-positions was observed, which indicates that the process is racemization free.

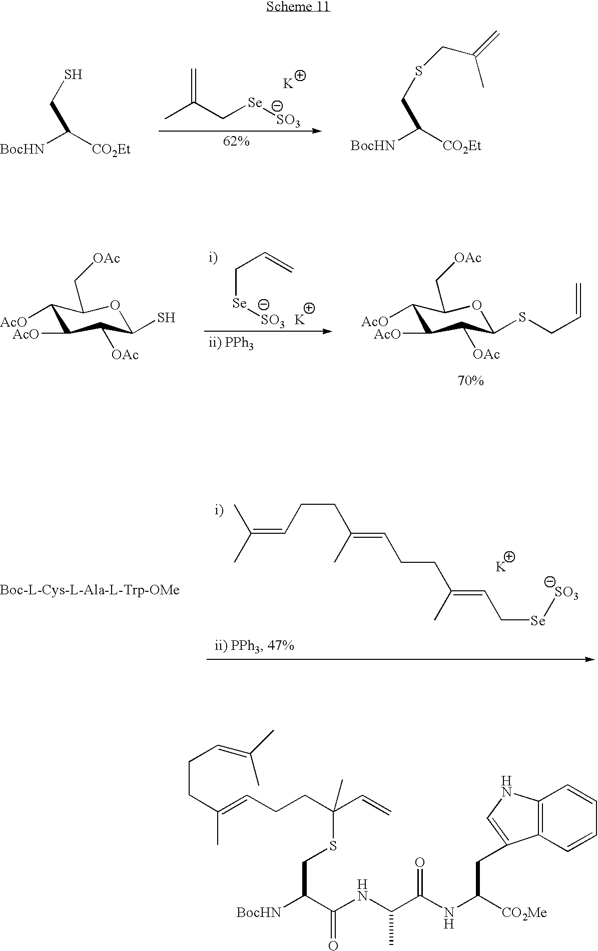

Difficulties in the synthesis of tertiary allylic Se-Bunte salts, such as would be capable of delivering the farnesyl chain in its linear unrearranged form, led us to reconsider the desulfurative rearrangements of allyl alkyl disulfides. In particular it was reported⁹⁹ that rate constants for the desulfurative rearrangement were between 0.7×10⁻⁴ and 8.6×10⁻⁴ s⁻¹ in benzene at 60° C. when R¹,R²=H and R³,R⁴=alkyl, whereas when R¹,R²=alkyl and R³,R⁴=H the rate constants varied between 1.4×10⁻² and 1.9×10⁻² s⁻¹ under the same conditions (Scheme 12). In other words the tertiary allylic systems rearrange almost two orders of magnitude faster than the primary allylic substrates.

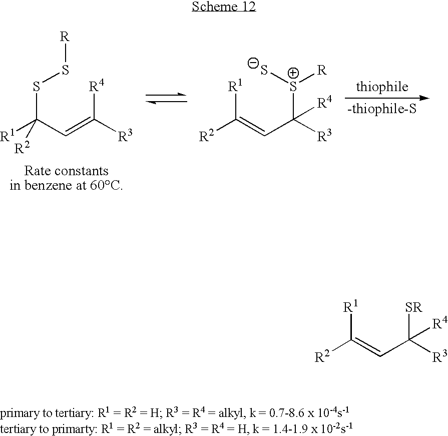

We reasoned further that the separation of charge as the reaction proceeds from the disulfide to the thiosulfoxide would lead to an enhanced concentration of the thiosulfoxide, effectively resulting in increased reaction rates, in more polar solvents. On this basis a model secondary allylic thiol was synthesized (Scheme 13) by the allylic xanthate rearrangement, a reaction that proceeds via a [3,3]-sigmatropic rearrangement.¹⁰¹⁻¹⁰⁶ This thiol was then converted to the 5-nitro-2-pyridyl disulfide in the standard manner for conjugation to a cysteine derivative, which was not isolated but treated with triphenylphosphine at room temperature in methanol/acetonitrile leading to the formation of the desired desulfuratively rearranged product at room temperature (Scheme 13). As anticipated on the basis of related [2,3]-sigmatropic rearrangements,¹⁰⁷⁻¹¹⁰ with their chair-like transition states, the product was obtained exclusively in the form of the trans-isomer.

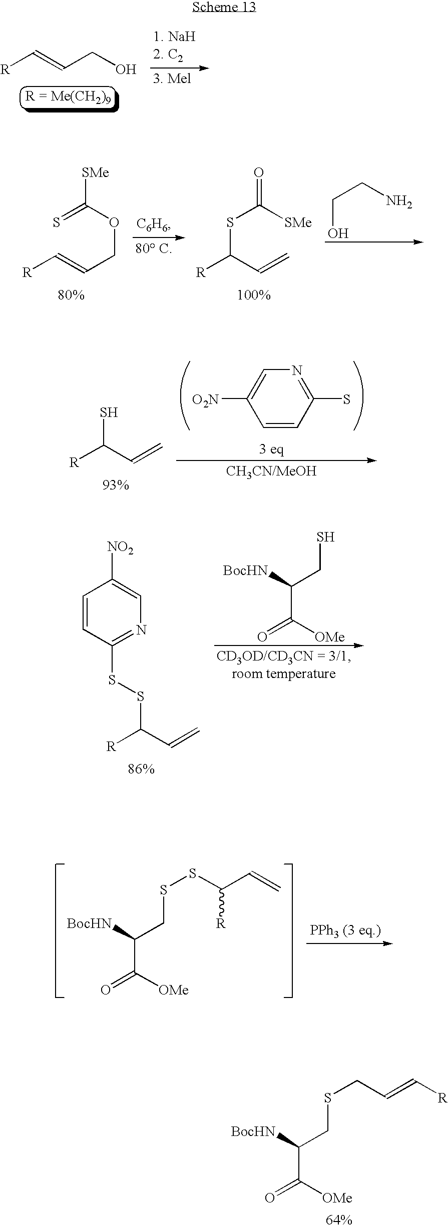

A number of examples were then carried out, some of which are illustrated in Scheme 14. All secondary and tertiary allylic thiols were readily prepared in high yield by the xanthate rearrangement protocol. We have used mixed disulfides with either 2-mercaptopyridine, 5-nitro-2-mercaptopyridine, or 2-mercaptobenzothiazole interchangeably, and find that all work well in these reactions. The ability to use the different heterocycles affords us flexibility and is most useful in avoiding the occasional difficult separation of the ligated product from the heterocyclic byproduct. Once again experiments conducted in methanol D₄ established the absence of racemization in these peptide-based experiments, all of which proceeded smoothly at room temperature.

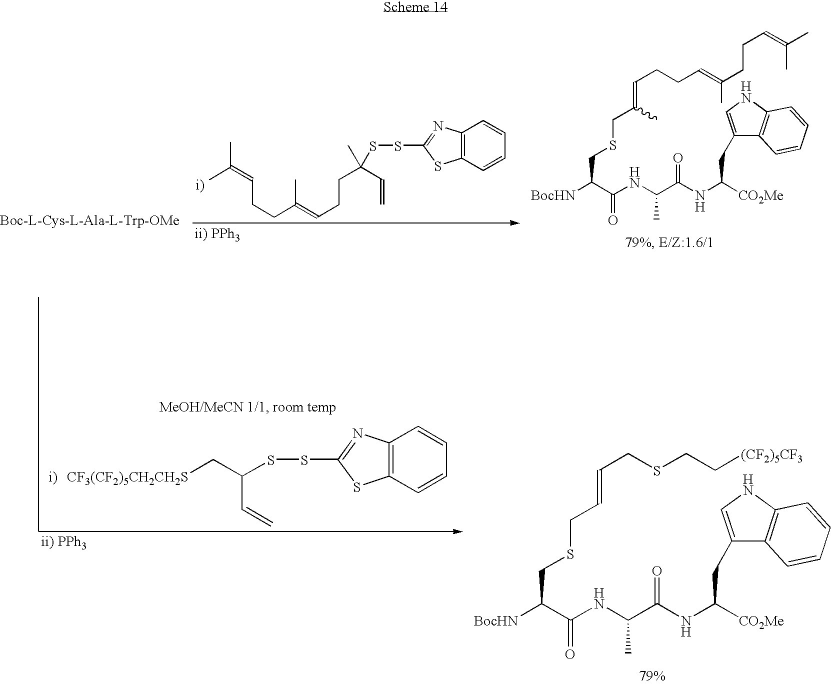

Overall the selenosulfide and disulfide chemistries, while conducted under essentially the same conditions, nicely complement each other as is evident from Schemes 11, and 14, with the introduction of the farnesyl chain in its rearranged form from the Se-Bunte salt, and in its more common linear form from the disulfide.

To date, in order to establish the functional group compatibility of the Se-Bunte salt/deselenative ligation we have successfully carried this reaction out on cysteine in the presence of all nineteen other proteinogenic amino acids. Yields were essentially unaltered in the presence of free carboxylic acids, amines, phenols, imidazoles, guanidines, indoles, alcohols, and sulfides. The very widespread use of the classical disulfide ligation, even for the lipidation of peptides and proteins,¹¹¹ ensures the compatibility of the new disulfide/desulfurative ligation with a very wide range of functional groups and with most types of bioconjugates and biopolymers. We emphasize that this new permanent ligation chemistry operates directly on cysteine itself, does not employ electrophilic species^{84, 85, 89} with their attendant selectivity problems, and only requires the addition of a phosphine, something it has in common with all variants on the Staudinger ligation.

The ability to operate directly and selectively on cysteine in this manner distinguishes this chemistry from other recent methods for “cysteine” functionalization, which require the prior synthesis of peptides containing, among others,³² dehydroalanine units,⁹⁰ β-haloalanine units,^{24, 29, 30} aziridine units,^{23, 92} and peptide synthesis with prefunctionalized cysteine derivatives,^{93, 94} and so more than compensates for the need to prepare the allylic disulfides and selenosulfides.

Both the selenosulfide and the disulfide chemistries result in the formation of S-allyl sulfides, a functional group which has much interesting chemistry of its own and which provides avenues for further functionalization. We are particularly interested in the allylic sulfur ylide rearrangement, which affords homoallylic sulfides via a [2,3]-sigmatropic shift, and which has found application in many areas of organic synthesis.¹¹²⁻¹¹⁴ We favor the Doyle-Kirmse modification of this reaction in which the allylic sulfur ylide is generated by the reaction of the allylic sulfide with a diazoalkane and a metal catalyst, as no external base is required and because the sequence proceeds around room temperature.¹¹⁵⁻¹²³ In addition, it has recently been demonstrated that several transition metal-catalyzed reactions may be conducted in aqueous solution on protein substrates.^{33, 124, 125} Most pertinently, it has been demonstrated that diazoalkane-derived rhodium carbenoids may be employed to functionalize tryptophan residues in proteins.¹²⁶

As proof of concept we have prepared the carbohydrate-based diazoacetamide 2 from the corresponding glycosyl amine 1 and have demonstrated its coupling to an S-allyl cysteine derivative with catalysis by dirhodium tetraacetate in dimethoxyethane at room temperature (Scheme 15). The product, the neoglycoconjugate 3, was obtained in an unoptimized 48% yield, as a 1:1 mixture of stereoisomers starting from stoichiometric quantities of the diazoamide and the allyl sulfide. In the context of peptide and protein modification, the question of compatibility of this chemistry with the tryptophan alkylation described by Francis obviously arises.¹²⁶ However, it having been previously demonstrated by Rainier in the context of the synthesis of quaternary thioindolines that allylic sulfides are more reactive than indoles towards rhodium carbenoids,¹²⁷ It appears that the sulfur ylide is formed more rapidly than attack at tryptophan.

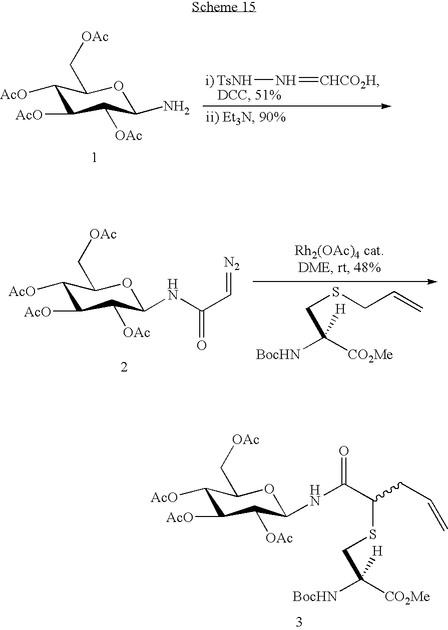

### Native Chemical Ligation

In order to extend the scope of native chemical ligation to phenylalanine, a protocol for the synthesis of a suitable β-mercaptophenylalanine derivative was devised. Rather than undertake an asymmetric synthesis of such a derivative, the functionalization of phenylalanine itself by means of free radical bromination as reported initially by Easton was investigated.^{128, 129} Thus, it had been demonstrated that the use of the powerfully electron-withdrawing N-phthalimido group directed, NBS-mediated bromination of phenylalanine preferentially to the β-position, resulting in the formation of N-phthalimido-β-bromophenylalanine derivatives in high yield.^{130, 131} Practical considerations, most notably the need for the subsequent removal of the nitrogen protecting group, suggested that the N,N-di-Boc system be explored as a replacement for the phthalimido derivative. We were pleased to find that treatment of 4,¹³² obtained from phenylalanine methyl ester by reaction with Boc₂O in the presence of DMAP,¹³³ with N-bromosuccinimide in tetrachloromethane at reflux, provided an intermediate β-bromophenylalanine derivative. On treatment with silver nitrate in acetone, this afforded the crystalline N-Boc oxazolidinone 5 in 60% yield for the two steps (Scheme 16). Treatment with cesium carbonate in methanol then provided the β-hydroxphenylalanine derivative 6, and an overall improved, shortened synthesis of this amino acid found as a component of numerous antibiotic substances.

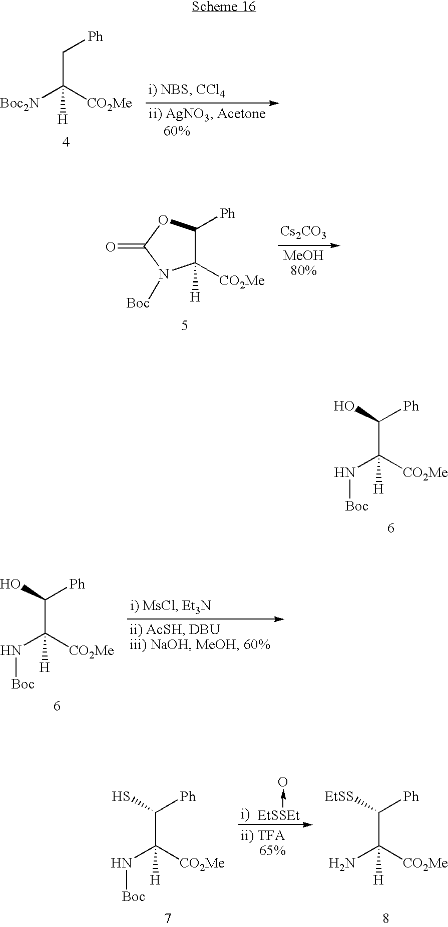

Mesylation, displacement with thiolacetic acid, and saponification then afforded the β-mercaptophenylalanine derivative 7. To protect against oxidation to the corresponding disulfide, this thiol was treated with diethyl thiosulfinate, itself readily obtained from diethyl disulfide with m-chloroperoxybenzoic acid,¹³⁴ to provide the S-ethylthio derivative from which the Boc group was removed in the standard manner to afford amine 8 (Scheme 16).

Proof of principle of the native chemical ligation was achieved when disulfide 8 and the N-Cbz glycine S-ethyl ester 9 were stirred in a mixture of 0.1 M tris buffer and acetonitrile at room temperature in the presence of excess sodium 2-mercaptoethylsulfonate, when the dipeptide 11 was obtained in 75% isolated yield (Scheme 17). Under the same conditions of concentration only negligible coupling was observed when the β-mercaptophenylalanine was replaced by phenylalanine methyl ester itself. Treatment of dipeptide 11 with nickel boride, generated in situ from nickel chloride and sodium borohydride, finally afforded the simple dipeptide 13. Exactly analogous results were observed on coupling of 8 to methionine 10. As anticipated it was possible to reduce the benzylic carbon-sulfur bond in the mercaptophenyl residue in 12 without desulfurization of the more robust methionine chain (Scheme 17).

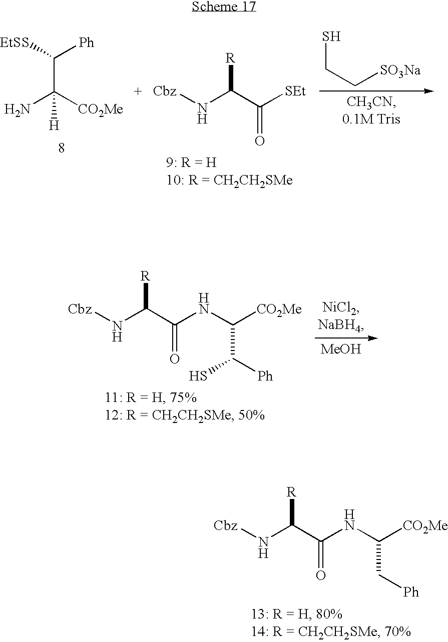

### Functionalization of Thiols by Sigmatropic Rearrangements

In a further demonstration of the power of the methods developed for the introduction of lipids to cysteine, the octapeptide 15 (SEQ ID NO: 1), which represents the C-terminal octapeptide of N-Ras,¹³⁸ is prepared by manual solid phase peptide synthesis using standard Fmoc techniques¹³⁵ and the Ellman safety catch linker.^{136, 137} This protein, after farnesylation at cysteine by the farnesyl transferase enzyme,¹³⁹ plays a major role in the regulation of many biological processes including cell signaling, differentiation and growth. The synthesis of S-farnesylated Ras peptides has attracted considerable attention, with previous approaches involving peptide synthesis with prealkylated cysteine residues,^{32, 93, 94} and nucleophilic attack by farnesyl thiol on electrophilic units in peptides.^{23, 29, 90, 92} While each of these methods has been successful to some extent, they suffer from the obvious problems of requiring a separate peptide synthesis for every lipid to be introduced, or of peptide synthesis with the incorporation of specific electrophilic units, which are not always straightforward to prepare. The present approach (Scheme 18) produces the farnesylated peptide 17 (SEQ ID NO: 2), does not require the incorporation of special amino acid building blocks, and permits the introduction of a variety of lipids simply by changing the allylic disulfide or selenosulfide. This protocol, with diversification as the final step, offers a considerable improvement in efficiency when multiple lipids are to be investigated. This flexibility is illustrated by incorporation of both of the physiologically relevant lipids, farnesyl and geranylgeranyl.

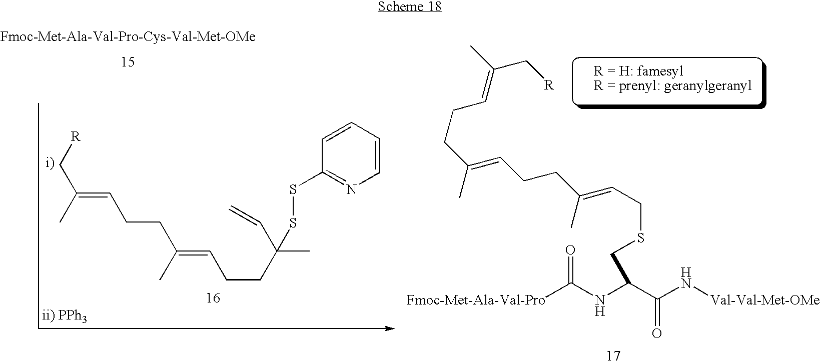

Turning to the synthesis of glyco- and neoglycoconjugates, a series of donors suitable for the preparation of neoglycoconjugates, are synthesized to demonstrate the facility of the ligation by joining them to short peptides. Acetobromoglucose 18, which is glycosylated under Koenigs Knorr conditions with an excess of cis-2-butene-1,4-diol gives the β-glycoside 19, whose stereochemistry is anticipated on the basis of neighboring group participation by the 0-2 acetate group. This substance is then converted to the monothiocarbonate under the Robins conditions,¹⁴⁰ subjected to the allylic “xanthate” rearrangement giving 20, and selectively hydrolyzed with hydrazine acetate to give the thiol 21.¹⁴¹ Finally, this thiol is activated for ligation by conversion to the pyridyl disulfide 22 by reaction with dipyridyl disulfide (Scheme 19). In parallel, the thiocarbonate 20 is subjected to saponification with sodium methoxide to give a fully deprotected glycosyl thiol 23, and, after disulfide formation, the donor 24.

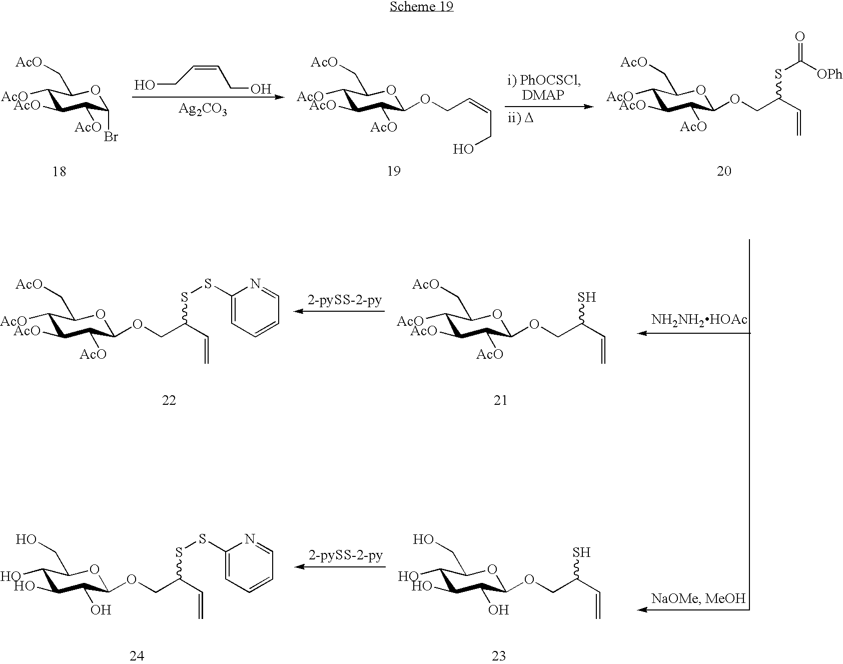

This first generation approach to neoglycoconjugate donors is based on the preliminary results outlined in Schemes 13 and 14, and on classical glycosylation chemistry. However, in the interest of synthetic efficiency, a second generation approach is developed to reduce the number of steps on the carbohydrate. Thus, monosilylated 2-butene-1,4-diol 25,¹⁴² is taken through the steps of Scheme 20 to provide a glycosyl acceptor 27. The coupling of this acceptor to acetobromoglucose is then investigated and provides a much shorter route to the two neoglycoconjugate donors 22 and 24.

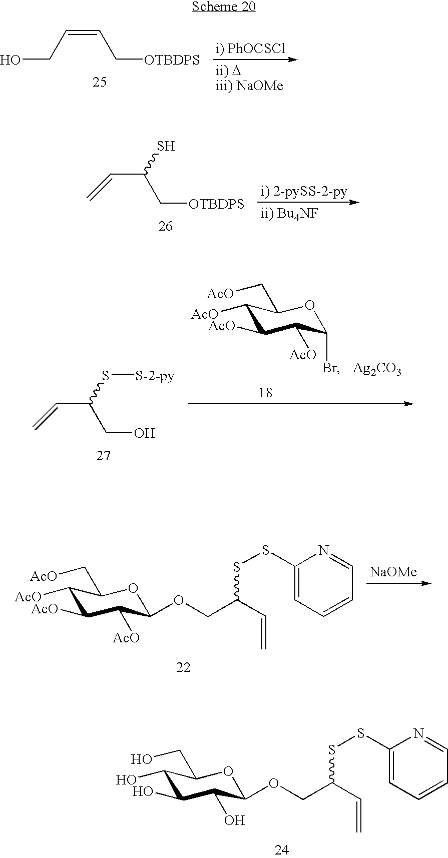

In Schemes 19 and 20, the Koenigs Knorr reaction is employed to prepare the glycosidic bond because of its well-known high propensity to afford the trans-glycoside and because of its operational simplicity, which makes it attractive to the non-carbohydrate specialist. We note that at the time of the last comprehensive survey, for the year 1994,¹⁴³ the Koenigs Knorr reaction was still the most widely employed glycosylation reaction, followed by the Schmidt trichloroacetimidate and then the thioglycoside methods. However, should the need arise, other glycosylation protocols such as the Schmidt trichloroacetimidate reaction can be employed.¹⁴⁴ Similarly, for illustrative purposes, the well-known 2-pyridyl sulfides have been selected, but the 5-nitro-2-pyridyl sulfides and the 2-benzothiazolyl sulfides can be used, both of which have been shown to function well in preliminary studies (Schemes 13 and 14).

With two neoglycoconjugate donors in hand, attachment to cysteine in the simple glutathione derivative 25 is checked,¹⁰⁰ as set out in Scheme 21. On the basis of the preliminary results presented in Schemes 13 and 14, i) the initial ligation proceeds rapidly at room temperature; ii) the desulfurative rearrangement proceeds at room temperature in the presence of triphenylphosphine to give 26 and 27, and iii) the rearrangement is completely trans-selective.

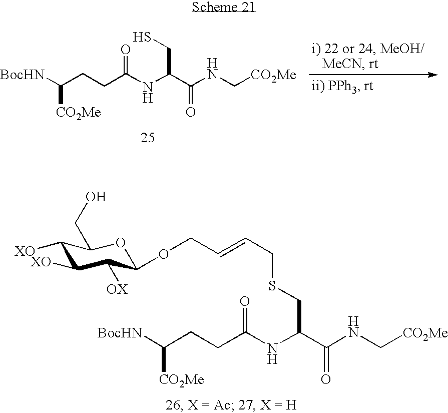

A set of neoglycoconjugate donors, derived from α and β-galactose, β-cellobiose, β-N-acetylglucosamine, β-chitobiose, α-N-acetylgalactosamine and α-sialic acid is prepared, covering all major types of linkage found in biologically active glycosylated peptides and proteins.^{1, 4, 11, 14, 145-149}

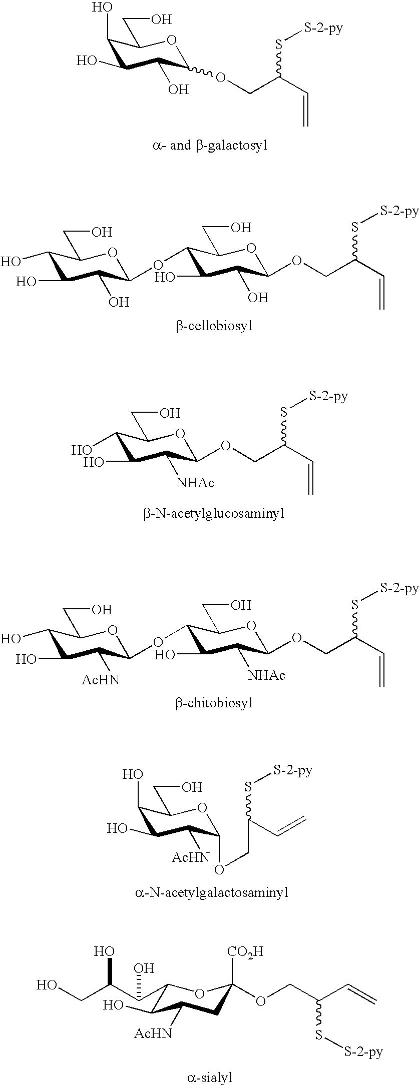

These neoglycoconjugate donors are coupled to a selection of short peptides, all of which are commercially available from Bachem, and which are selected so as to illustrate the compatibility of the new ligation with the broadest range of typical peptide side chains, rather than for any particular biological activity. For example, the formation of neoglycoconjugates of H-Cys-Gln-Asp-Ser-Glu-Thr-Arg-Thr-Phe-Tyr-OH (SEQ ID NO: 3), a decapeptide fibronectin fragment,^{150, 151} that contains the side chain amide group of asparagine, the side chain of aspartic and glutamic acids, the hydroxyl group of threonine, the phenolic hydroxyl group of tyrosine, and the guanidine of arginine, is investigated to provide a significant test for the new ligation. Each of these individual amino acids has been demonstrated to be compatible with the ligation reaction in preliminary experiments, and this compatibility translates to the more complex peptide environment. In particular we stress the very widespread application of the simple disulfide ligation (Scheme 7) in peptide and protein chemistry.⁸¹⁻⁸⁵ This chemistry is conducted in an aqueous environment, and the products are isolated by reverse phase HPLC, and characterized by whichever of MALDI-TOF or electrospray mass spectrometry that proves the most suitable.

In addition to the neoglycoconjugates prepared with the above series of donors, with their trans-2-butenyl spacer, a series of donors designed to introduce a glycosidic bond directly onto the sulfur of cysteine are investigated. Thus, for example, reaction of triacetyl-D-glucal 28 with hot aqueous dioxane will provide, by means of the Ferrier reaction,¹⁵² an anomeric mixture of the rearranged glycols 29. The formation of the trans-enal 30, which plagued early work in this area, may be suppressed by working in the presence of hydroquinone, or in the dark.¹⁵³ Treatment with phenyl thionochloroformate provides the anomeric thiocarbonates 31, which are separated into their constituent anomers before heating to bring about the [3,3]-sigmatropic rearrangement to the 3-deoxy-3-thioglycals 32 and 36. Selective cleavage of the thiocarbonates is followed by disulfide formation with 2,2′-dipyridyl disulfide, or its equivalent, in the standard manner. Saponification under Zemplen conditions provides the deprotected donors 35 and 39 (Scheme 22).

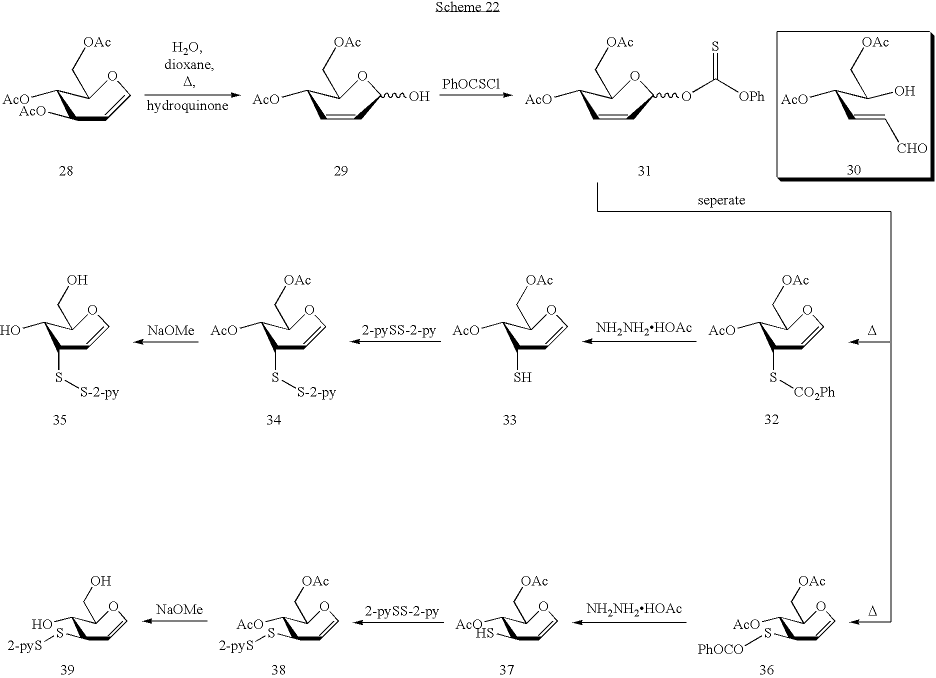

These four glycosyl donors are used in a number of coupling reactions. First, the protected forms 34 and 38 are used to glycosylate cysteine and small di- and tripeptides, so as to determine the optimum reaction conditions. The ribo-configured donor 34 proceed to give an α-linked S-glycosyl cysteine derivative 40, and that the arabino-isomer 38 afford a β-linked cysteine derivative 41 (Scheme 23). The stereospecificity of these reactions is predicated on the sigmatropic rearrangement nature of the ligation as established by the preliminary results.

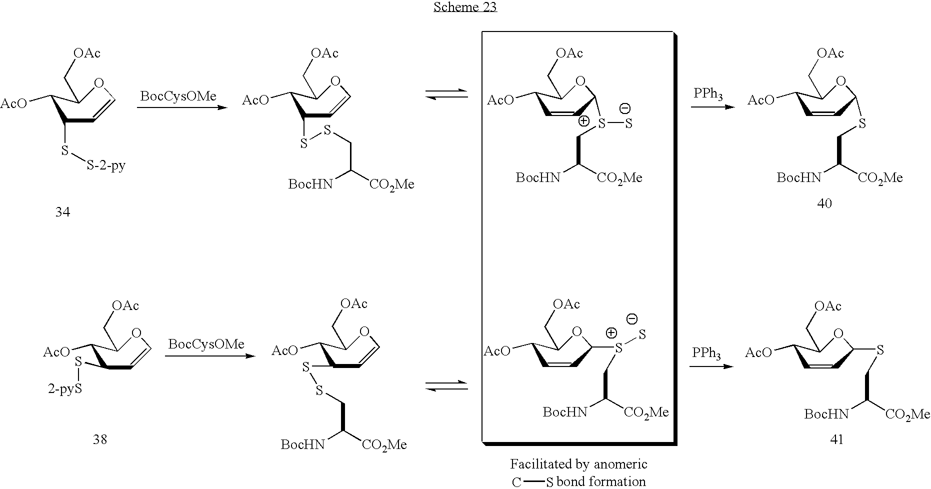

The sigmatropic rearrangement is facilitated by the formation of the anomeric C—S bond, which is worth about 1.5 kcal·mol⁻¹ in 2-methylthiotetrahydropyran,^{154, 155} in much the same way that a 2-O-acyl group migrates to the anomeric center in the rearrangement of anomeric radicals (Scheme 24).¹⁵⁶⁻¹⁵⁸

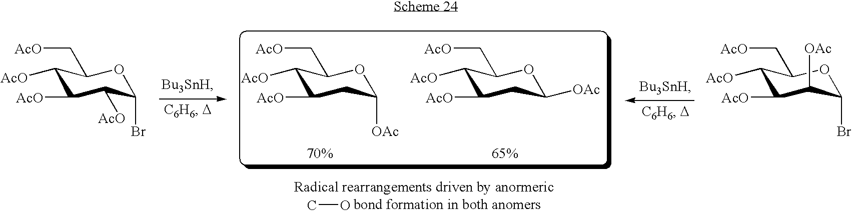

The 2,3-unsaturated nature of the products of these couplings provides the opportunity for further manipulations, such as diimide reduction to simple 2,3-deoxy glycosides (Scheme 25).¹⁵⁹ Based on the work of O'Doherty,¹⁶⁰ Feringa,¹⁶¹ and Lee¹⁵⁹ with the analogous O-glycosides, osmoylation of the α-isomers is highly selective for the manno-product, while the β-series affords the allo-isomer (Scheme 25).

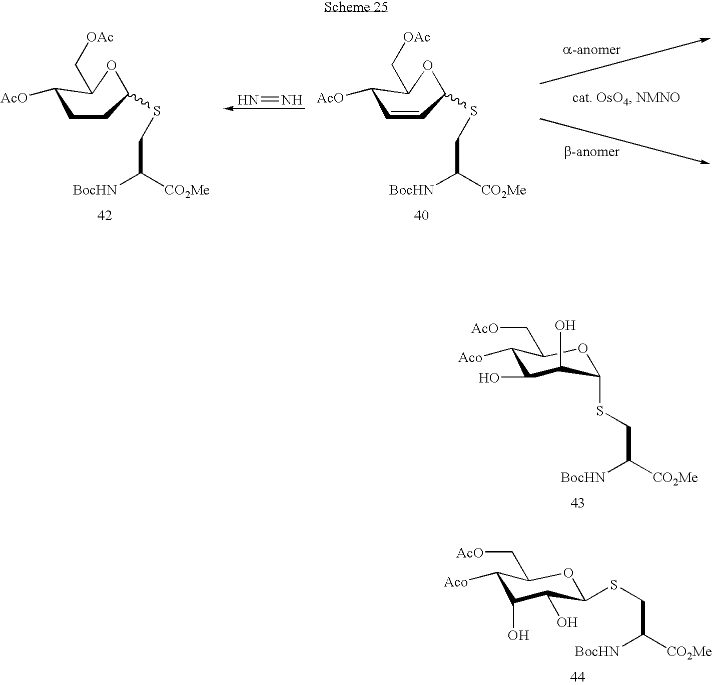

With the basic chemistry established with the protected donors 34 and 38, the viability of the chemistry in aqueous solution is examined with the unprotected donors 35 and 39 and short commercial peptides such as the H-Cys-Gln-Asp-Ser-Glu-Thr-Arg-Thr-Phe-Tyr-OH (SEQ ID NO: 3) discussed above. The method is also demonstrated by coupling a donor 45 prepared from galactal, and di- and trisaccharide donors 46 and 47 prepared from a 6-O-triphenylmethyl derivative of 39 and acetobromoglucose and acetobromcellobiose, with short commercial peptides.

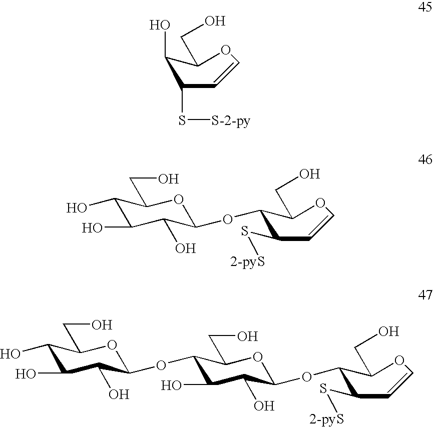

The use of exocyclic glycals, which are readily obtained by olefination of glycuronolactones is also explored.¹⁶² Thus, for example, the readily prepared exocyclic glycal 48 is reduced to the corresponding alcohol and converted to the corresponding thiol by Mitsunobu reaction with thioacetic acid^{141, 163, 164} and saponification. Formation of the pyridyl disulfide 49, coupling to cysteine, and final triphenylphosphine-promoted desulfurative rearrangement ultimately provides the glycosylated peptide. Final cleavage of the two acetonide protecting groups is achieved with trifluoroacetic acid, conditions which are fully compatible with peptides, to give the novel glycosylated peptide 50 (Scheme 26). The α-glycoside is formed selectively in this particular example because of the hindrance of the β-face by the 2,3-O-isopropylidene group. As in Scheme 23, an extra driving force for the desulfurative rearrangement is derived from the formation of the anomeric C—S bond. Of course, if this effect is insufficient to enable the desulfurative rearrangement at room temperature, our analogous selenosulfide rearrangement can be used, with its established ability to afford tertiary allylic sulfides at room temperature.¹⁰⁰

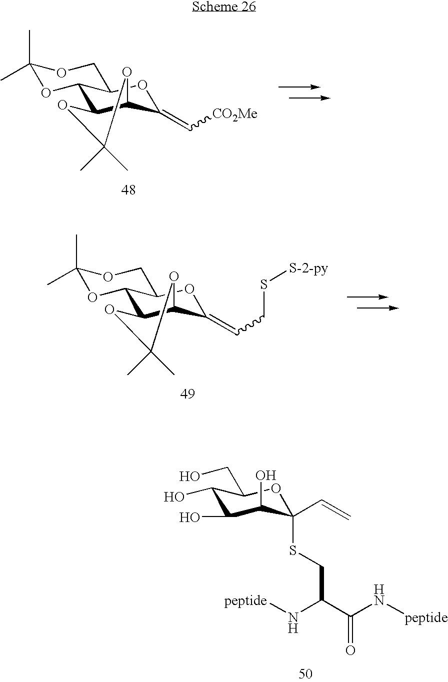

The present invention also relates to use of the new ligation reaction for the attachment of polyethyleneglycol chains to cysteines and other thiols. The attachment (pegylation) of polyethyleneglycol (PEG) chains to drugs, especially to peptide and protein-based drugs of which more than 80 are currently marketed in the USA, has many advantages.^{85, 170, 171} Aqueous solubility is improved, antigenicity of protein-based drugs is reduced, rapid kidney clearance is cut down, and circulating half-life is extended.^{85, 170, 171} Many methods have been devised for pegylation, most of which involve the use of peg-based electrophiles that suffer from poor chemoselectivity.^{85, 170-175} Pegylation by means of the classical disulfide linkage has been developed,^{170, 176} but this is obviously reversible in the presence of glutathione and therefore has limited application in drug delivery.

The present invention, on the other hand, provides for synthesis of Peg reagents for the permanent and selective ligation of cysteine residues. This is achieved through the synthesis of an amine analog 58 of the alcohol 27, whose preparation is described in Scheme 20. The amine is activated in the form of its chloroformate 59, or a related activated derivative, such as the N-hydroxysuccinimide-based carbamate 60, and is coupled to commercial monomethylpoly-ethylene glycol (MPEG) in the standard manner. The use of an excess of the reagent 59 ensures high conversion, and isolation is achieved by precipitation of the MPEG from solution by addition of ether.^{177, 178} Analysis is conducted primarily by NMR spectroscopy using the terminal methyl group of the PEG chain as an internal standard for integration,^{177, 178} with confirmation by MALDI-TOF mass spectrometry. Finally, the MPEG-based disulfide is used to derivatize standard commercial peptides. Following dechalcogenative rearrangement with triphenylphosphine, or a water soluble phosphine, final purification is achieved by size exclusion chromatography, with confirmation of structure by MALDI-TOF mass spectrometry (Scheme 27).

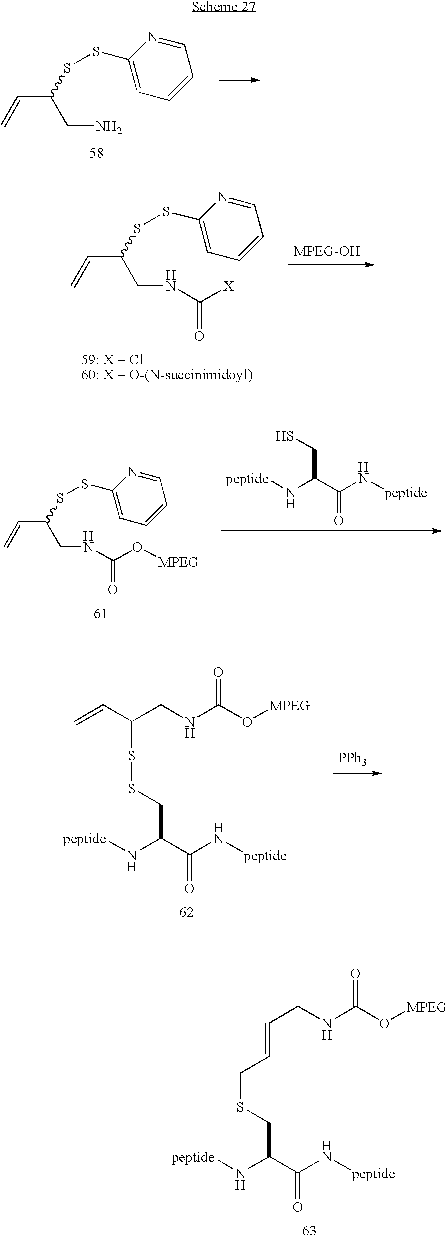

Variations on the above approach can also enable the permanent labeling of peptides and proteins, selectively through cysteine, with spin labels,¹⁷⁹ for irreversible biotinylation¹⁸⁰ of cysteine residues, for the attachment of fluorescent markers,¹⁸¹⁻¹⁸³ and of fluorous affinity labels.¹⁸⁴

As noted above, the commercial decapeptide fibronectin fragment H-Cys-Gln-Asp-Ser-Glu-Thr-Arg-Thr-Phe-Tyr-OH ^{150, 151} (SEQ ID NO: 3) is a convenient substrate with which to evaluate the applicability of the present cysteine derivatization protocols based on dechalcogenative allylic rearrangements of allylic disulfides and selenosulfides. However, a set of decapeptides designed and employed by Dawson can also be used to determine the functional group compatibility of native chemical ligation.¹⁸⁵ Thus, Dawson and co-workers synthesized the pentapeptide Cys-Arg-Ala-Asn-Lys (CRANK; SEQ ID NO: 4), with a N-terminal cysteine, and coupled it by standard native chemical ligation to the pentapeptide thiol ester Leu-Tyr-Arg-Ala-Xaa-SR (LYRAX-COSR; SEQ ID NO: 5) to give the decapeptides Leu-Tyr-Arg-Ala-Xaa-Cys-Arg-Ala-Asn-Lys (SEQ ID NO: 6).¹⁸⁵ This particular set of peptides was chosen as it was considered that, with variation of the Xaa residue, it would permit rapid assay of the applicability of native chemical ligation with formation of some of the more challenging Xaa-Cys bonds in the presence of some of the more reactive amino acid side chains. This same set of peptides is ideal testing grounds for our cysteine lipidations, various glycosylation and neoglycosylation sequences, and the PEGylation reactions. Thus, the Dawson synthesis of the CRANK pentapeptide is repeated using the manual solid phase peptide synthesis Boc-based protocols described. Similarly, the Dawson synthesis of several LYRAX-COSR (SEQ ID NO: 5) pentapeptides is repeated in which X is Phe, Trp, Val, Pro and Leu, and LYRAX-COSR (SEQ ID NO: 5) is coupled to CRANK (SEQ ID NO: 4) by native chemical ligation to give a set of five decapaptides, with relatively bulky side chains adjacent to the cysteine residue. These peptides are prepared on 0.25 mmol scale, as described by Dawson, and are used to test each of the cysteine functionalization reactions in the presence of reactive amino acid side chains and in hindered steric environments. All peptides are purified by RP-HPLC, both before and after the cysteine functionalization step, and analyzed by NMR spectroscopy and ESI and MALDI-TOF mass spectrometry.

The use of the Doyle-Kirmse reaction is also investigated for the modification of the various S-allylic peptides prepared by the dechalocogenative rearrangement of allylic disulfides and selenosulfides. Thus, in addition to the glucose-based diazoacetamide 2 described in Scheme 15, a range of diversely functionalized diazoalkanes and diazocarbonyl compounds are prepared and subjected to rhodium catalyzed couplings to our various S-allyl products. Some of these diazo derivatives are illustrated in Scheme 28. All of these compounds are prepared by well-known methods,¹⁸⁶ and by variation on the theme illustrated in Scheme 15.

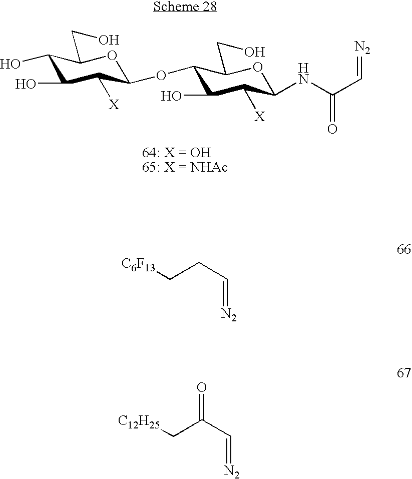

### Asparagine Glycosylation

Glycosylation at nitrogen of asparagine is a critical step in the synthesis of any N-linked glycoprotein.^{11, 187} The problem may be approached either by formation of the N-glycosyl bond (Scheme 29, path a), i.e. by an N-glycosylation reaction, or by formation of the N—CO bond (Scheme 29, path b) in an amide forming reaction. Both approaches are problematic. The glycosylation route suffers from the poor nucleophilicity of the amide

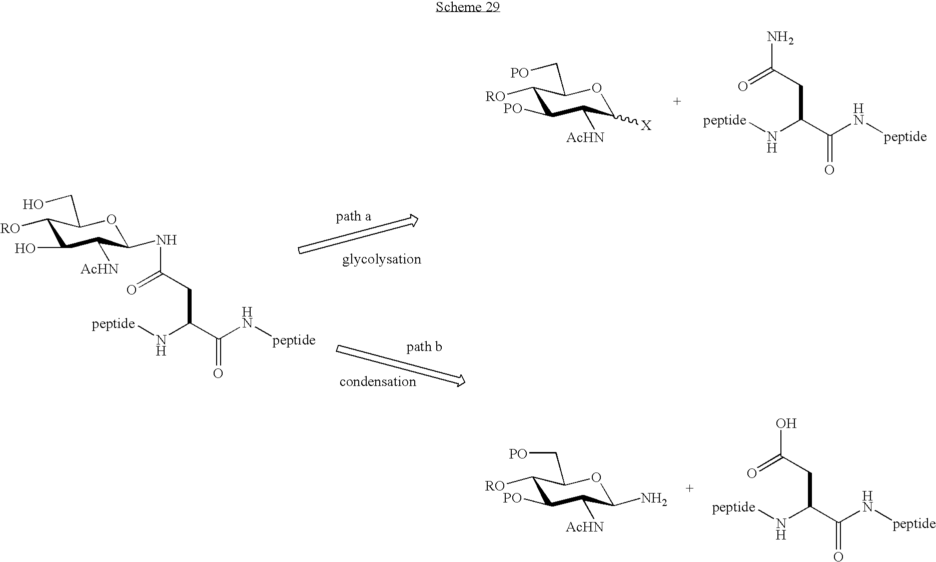

nitrogen, the propensity of amides to undergo reaction on oxygen leading to imidates, and the ever present problem in any glycosylation reaction of anomeric stereoselectivity.¹⁸⁸ The amide bond forming route suffers from the instability of glycosyl amines, and their tendency toward anomerization, and the reduced nucleophilicity of anomeric amines as compared to typical primary amines. Improvements on both approaches are being constantly sought,^{6, 21, 80, 189} and new methods are under development, as for example the traceless Staudinger reaction depicted in Scheme 6.^{6, 78, 79} Evidently, this is a far from solved problem that is a significant bottleneck in any project targeted at large scale synthesis of N-glycopeptides for studies on synthetic vaccines.¹⁹⁰ The present invention utilizes two approaches to this problem. The first is based on a variant of Fukuyama's creative use of nitrobenzenesulfonamides as amine activating an protecting groups, and the second is a variation on the theme of native chemical ligation.¹⁹¹

As described by Fukuyama and co-workers the nitrobenzenesulfonyl and dinitrobenzenesulfonyl moieties are very versatile protecting groups for amines. Their strong electron-withdrawing nature aids deprotonation of the sulfonamide NH and thereby facilitates alkylation at nitrogen. The highly electron-deficient nature of the nitrobenzenesulfonamide aromatic ring enables facile deprotection with nucleophilic thiols through a nucleophilic aromatic substitution and desulfonylation process, and this chemistry has seen wide application in a variety of contexts.¹⁹¹ A lesser known variant on this theme was introduced by Tomkinson and uses thio acids as nucleophile in the deprotection step.¹⁹² The reaction proceeds at room temperature in DMF in the presence of cesium carbonate and releases the amine in the form of an amide with the original thio acid (Scheme 30). It has yet to be determined whether the decomposition of the intermediate Meisenheimer complex 68 to the products is a concerted process with intramolecular acyl transfer, or if an intermediate S-acyl dinitrothiophenol derivative is involved. Dithioacids, dithiocarbamic acids, and hydroxamic acids have also been employed as nucleophiles when the products are thioamides, thioureas, and ureas, respectively.¹⁹³ The overall transformation resembles the chemistry of the Williams,^{48, 49, 52} Liskamp,⁵⁰ and Knapp groups in which thio or selenoacids⁵¹ are reacted with azides to amides (Scheme 3), but takes place under much less forcing conditions and is much more general in the range of R groups tolerated.

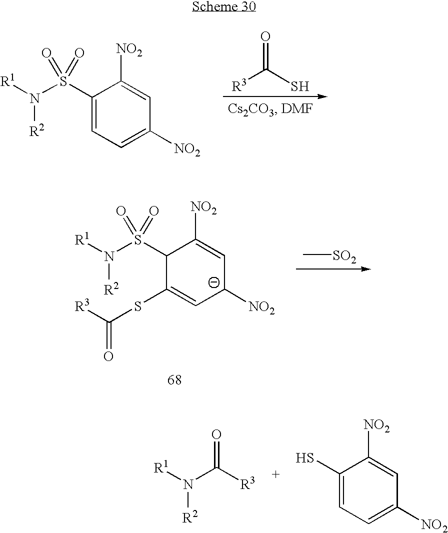

### Sulfonamide-Based Ligation.

A preferred method aspect of the present invention provides for ligation or derivatization of a peptide. The method comprises reacting sulfonamide (I) with peptide (II) to form ligated peptide (III):

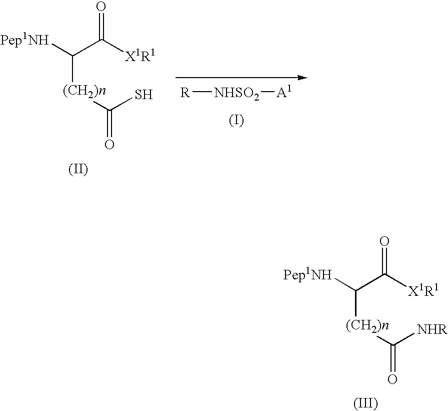

wherein R is an amino acid, a peptide, a monosaccharide, or a polysaccharide, optionally including one or more protecting groups on a nitrogen, oxygen, or sulfur substitutent thereof; X¹ is O or NH; R¹ is an alkyl group, an alkenyl group, an aryl group, an alkyl-substituted aryl group, an aryl-substituted alkyl group, an amino acid, or a peptide, optionally including one or more protecting groups on a nitrogen, oxygen, or sulfur substitutent thereof; A¹ is an electron deficient alkyl, aryl, or heteroaryl group; Pep¹ is an amino acid or a peptide, which optionally includes one or more protecting groups on a nitrogen, oxygen, or sulfur substitutent thereof; and n is 1 or 2 (preferably 1). In a preferred embodiment, X¹ is NH; and R¹ is an amino acid or a peptide, optionally comprising one or more protecting groups on a nitrogen, oxygen, or sulfur substitutent thereof, or an amino acid. In another preferred embodiment, X¹ is NH, and R¹ is a benzyl group or a C₁-C₄ alkyl group.

In some preferred embodiments, R is a peptide or an amino acid, optionally comprising one or more protecting groups on a nitrogen, oxygen, or sulfur substitutent thereof. In other preferred embodiments, R is a polysaccharide or a polysaccharide comprising one or more protecting groups on a nitrogen, oxygen, or sulfur substitutent thereof.

A¹ preferably is a nitro-substituted aryl group (e.g., a nitro-substituted phenyl group), a fluoroalkyl-substituted aryl group, an aryl tetrazole, or a pyridium group.

This method is applied to glycosyl sulfonamides and their coupling to aspartate side chains. Thus, in a proof of principle experiment, the direct glycosylation of sulfonamides on nitrogen by the Mitsunobu protocol is employed to form the requisite N-glycosyl sulfonamide 70, with separation of anomers at this stage as necessary. This protocol was established for the 2-nitrobenzenesulfonamides by the van Boom group,¹⁹⁴ and was subsequently employed successfully by other laboratories,¹⁹⁵ Once formed the N-glycosyl sulfonamide is configurationally stable.^{194, 195} This sulfonamide is then coupled with the aspartate derivative 72, which is prepared by the reaction of a side chain activated aspartate derivative with sodium hydrogen sulfide, to give the expected N-glycosyl asparagine 73 (Scheme 31).

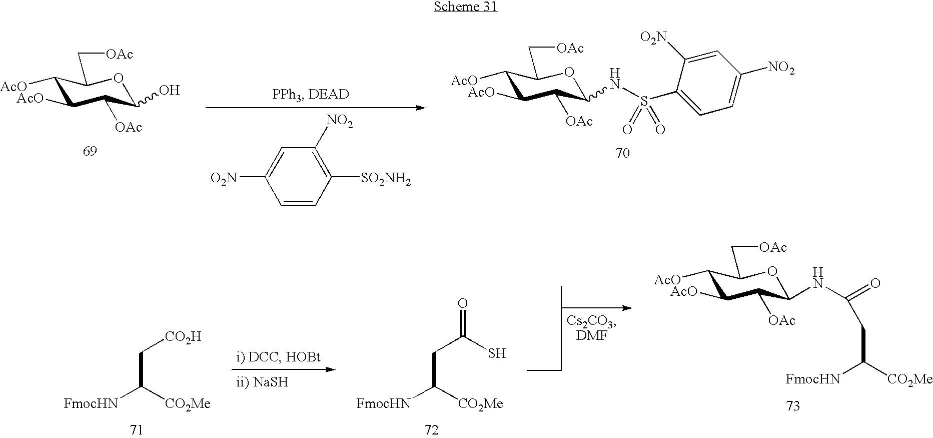

As a back-up protocol, the reaction of acetobromoglucose with sodium azide affords the well-known, configurationally-stable β-glucosyl azide 74, which is reduced with hydrogen on palladium charcoal to give the β-glycosyl amine (Scheme 32). This is immediately coupled with an excess of sulfonyl chloride to give the N-glycosyl sulfonamide 70. Chromatographic purification at this stage enables separation of any anomeric mixtures formed owing to scrambling at the level of the amine, a problem which is minimized by forming the amine from the azide by hydrogenolysis and its immediate use in the coupling reaction. For the 2-deoxy series it is also well-established that N-iodosuccinimide protomoted addition of sulfonamides to glycals results in the high yield formation of 2-deoxy-2-iodoglycosylsulfonamides.^{196, 197}

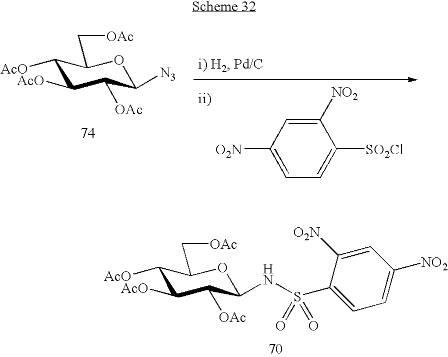

Having established the principle, and before proceeding to the application of this reaction in glycopeptide synthesis, the use of alternative aromatic ring systems for the nucleophilic aromatic substitution is investigated. We draw inspiration from the well-known Mukaiyama coupling reagents, the N-methyl-2-halopyridinium salts,¹⁹⁸ and from other condensation reactions involving a nucleophilic aromatic substitution step. In particular, attention is called to the variant of the Julia reaction, which employs 2-benzothiazolyl sulfones, and which was much improved by the introduction of the alternative 1-phenyl-1H-tetrazolyl-5-sulfones by Kocienski,¹⁹⁹ and the 3,5-bis(trifluoromethyl)phenylsulfones by Najera.²⁰⁰ Thus, a number of sulfonyl amides are prepared and coupled to pyranose 69 to give a range of N-glycosyl sulfonamides, some of which are depicted in Scheme 33, and these sulfonamides are allowed to react with thioacetic acid to give the product 78. All coupling reactions are carried out under a standard set of conditions, at room temperature, and are monitored by HPLC analysis or by UV monitoring of the released arene thiolate as appropriate.

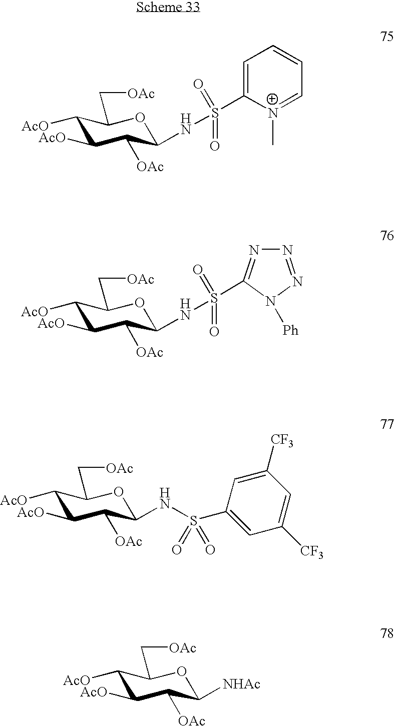

Once the optimal sulfonamide is determined, the ester protecting groups are stripped under standard Zemplen conditions (catalytic sodium methoxide in methanol) and the deprotected sugar 79 is coupled to the test thioaspartate 72 (Scheme 34). This coupling reaction is also conducted in the presence of the Fmoc derivatives of all twenty standard amino acids so as to determine functional group compatibility with the new chemistry. Cysteine, with its nucleophilic thiol group, is the most obvious source of problems and competing reactions but this is not catastrophic as the monothiocarboxylate group is significantly more nucleophilic. This is easily verified by carrying out the reaction of Scheme 34 in the presence of an equimolar quantity of cysteine, and is overcome if necessary by adjusting the conditions such that only the more acidic thioacid is deprotonated.

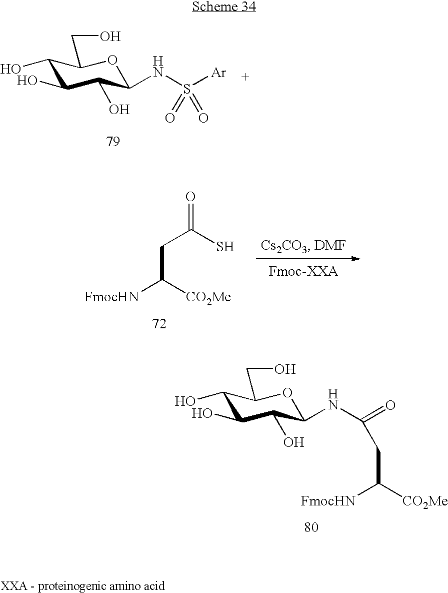

The present method is next applied to the glycosylation of a short peptide sequence (Scheme 35). This requires the synthesis of the target peptide sequence containing a thioasparate residue. In view of the importance of peptide thioesters in native chemical ligation, several improved methods have been developed recently for the synthesis of thioesters compatible with the preferred Fmoc strategy for peptide and glycopeptide synthesis²⁰¹⁻²⁰⁵. Thus, adapting the Kenner safety catch linker strategy,²⁰⁶ and especially its application to thioesters as described by Bertozzi and co-workers,²⁰³ benzenesulfonamide is coupled to Fmoc-L-Asp-O-t-Bu 81, followed by removal of the t-butyl ester with trifluoroacetic acid in the standard manner. This building block is then incorporated into the target peptide by the standard Fmoc strategy with which the acyl sulfonamide is fully compatible. Target peptides include, in order of increasing complexity with Xaa representing the thioaspartate residue, the Ser-Xaa-Leu-Thr-NH₂ (SEQ ID NO: 7) employed by Davis to test his recent asparagine glycosylation,⁸⁰ the Leu-Ala-Xaa-Val-Thr-NH₂ (SEQ ID NO: 8) favored by the Danishefsky group in establishing their glycopeptide constructs,¹⁹⁰ and the Gly-Asn-Xaa-Glu-Thr-Ser-Asn-Thr-Ser-Ser-Pro-Ser-NH₂ peptide (SEQ ID NO: 9) of the CD52 antigen from human lymphocytes and sperm whose glycosylation has been studied by Guo and co-workers.²⁰⁷ The peptides are constructed using Fmoc techniques in the solution phase for the tetra- and pentapeptides and on the peptide synthesizer located in the Protein Research Laboratory, a service facility of the campus Research Resources Center, using the building block 82. With the complete peptides in hand the acyl sulfonamide is activated for displacement under the Ellman conditions^{136, 137, 203} with iodoacetonitrile, and the activated form is displaced with sodium hydrogen sulfide to give the thioaspartate containing peptides ready for coupling with the glycosyl sulfonamide (Scheme 35). Initial work involves the simple model β-glucoside illustrated, but the method can be extended to include other representative monosaccharides, and complex oligosaccharides from oligosaccharide synthesis programs.^{46, 208, 209}

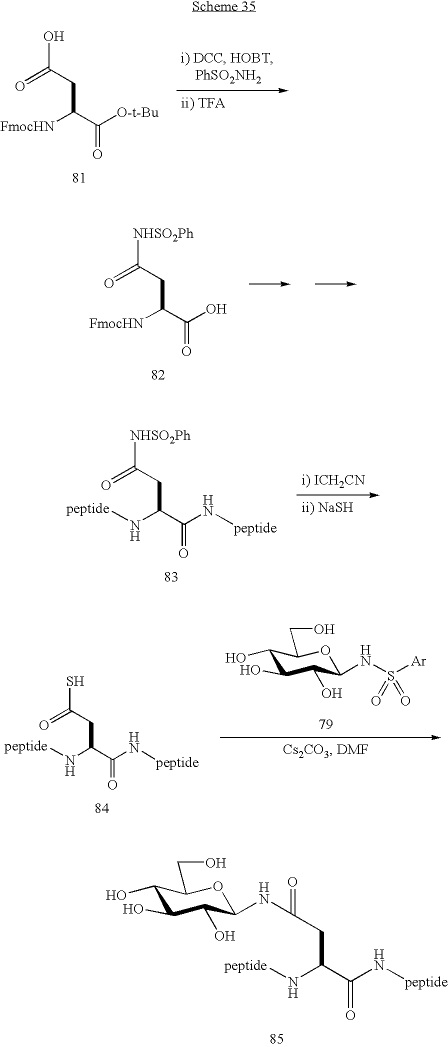

The second strategy investigated for the preparation of N-glycosyl asparagine derivatives is based on methods for native chemical ligation with cysteine surrogates developed by several groups,⁶⁸⁻⁷⁴ as described in Scheme 5. Thus, a series of amino disulfides, including 86 and 87 is prepared, and subjected to glycosylation reactions, for example with acetobromoglucose and silver catalysis, to give the corresponding N-glycosides 88 and 90. Saponification provides the corresponding unprotected sugars 89 and 90 in the standard manner. In designing this scheme, the o-nitrobenzyl type systems, developed by Marzini and co-workers for the native chemical ligation of peptides,⁷⁴ are selected as the final deprotection conditions are compatible with both peptidic functionality and with the presence of glycosidic bonds.

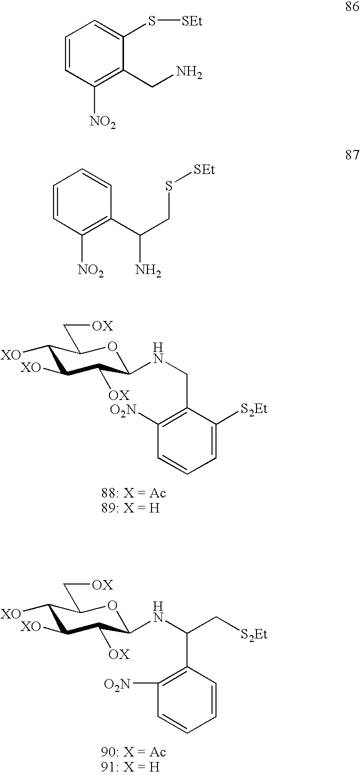

### Amino-Disulfide Mediated Peptide Glycosylation.

Another preferred method aspect of the present invention provides for glycosylation of a peptide. The method comprises reacting a N-glycosyl-o-nitrobenzylamino-disulfide compound (Glyc¹-NHZ) with peptidyl thioester (IV) in the presence of a thiol, and subsequent photolysis to afford glycosylated peptide (V):

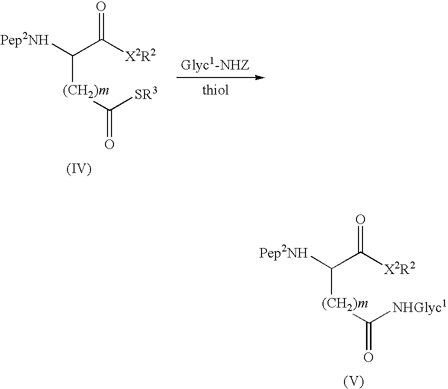

wherein X² is O or NH; R² is an alkyl group, an alkenyl group, an aryl group, an alkyl-substituted aryl group, an aryl-substituted alkyl group, an amino acid, or a peptide, optionally including one or more protecting groups on a nitrogen, oxygen, or sulfur substitutent thereof; R³ is an alkyl group, an alkenyl group, an aryl group, an alkyl-substituted aryl group, or an aryl-substituted alkyl group; Pep² is an amino acid or a peptide, which optionally includes one or more protecting groups on a nitrogen, oxygen, or sulfur substitutent thereof; Glyc¹ is a monosaccharide or a polysaccharide, optionally including one or more protecting groups on a nitrogen, oxygen, or sulfur substitutent thereof; Z is an o-nitrobenzyl-disulfide moiety; and m is 1 or 2 (preferably m is 1). In a preferred embodiment, R³ is C₁-C₄ alkyl or benzyl; preferably R³ is ethyl. The thiol preferably is a 2-mercaptoethanesulfonic acid salt.

In preferred embodiments, Glyc¹-NHZ can have one of the following structures:

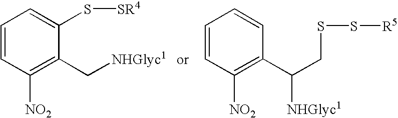

wherein R⁴ and R⁵ can be an alkyl group, an alkenyl group, an aryl group, an alkyl-substituted aryl group, or an aryl-substituted alkyl group.

As illustrated in Scheme 36 with the disulfide 91, glycosyl donors are coupled to aspartate thiol esters under the standard conditions of native chemical ligation to give the N-glycosyl asparagine derivatives. The preparation of the aspartate thiol esters follows the protocol set out in Scheme 35 for the corresponding thioacids, except that a thiol is substituted for the sodium hydrogen sulfide. Cleavage of the o-nitrobenzyl handle is finally be achieved photolytically,^{210,211} conditions which have recently been successfully employed in native chemical ligation,^{73, 74} and in glycoprotein synthesis.²¹ As described herein, the chemistry is first put on a firm basis with simple carbohydrate and amino acid derivatives before proceeding to more complex sugars and peptides of the type described above for the N-sulfonyl glycosyl amine chemistry.

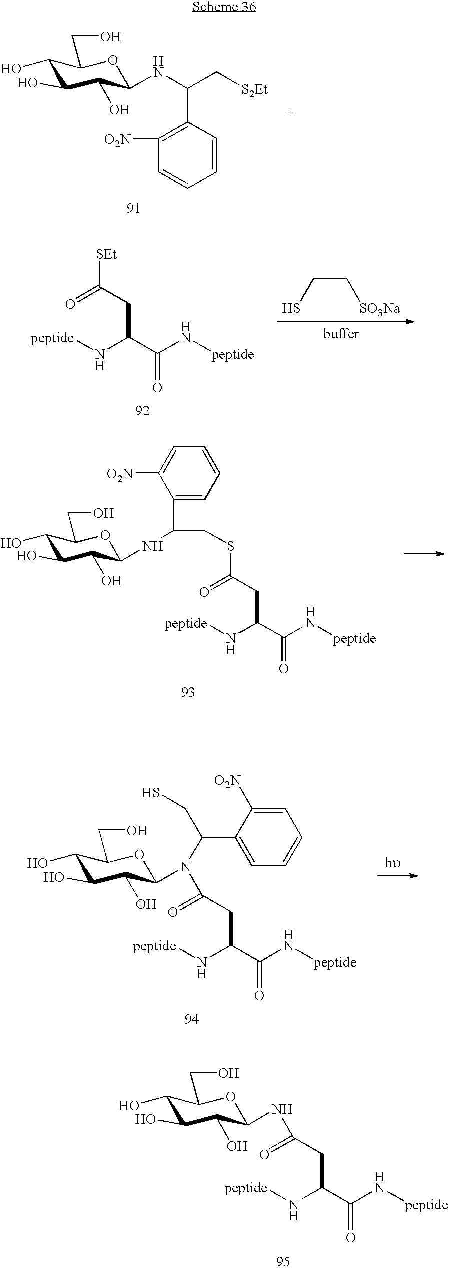

### Amino-Disulfide Mediated Native Chemical Ligation.

Yet another method aspect of the invention provides for native chemical ligation of a peptide at a phenylalanine, tyrosine or tryptophan residue, and comprises reacting peptidyl thioester (IV) with aminodisulfide (VI) in the presence of a thiol to afford mercapto peptide (VII); and optionally reducing (VII) to afford peptide (VIII):

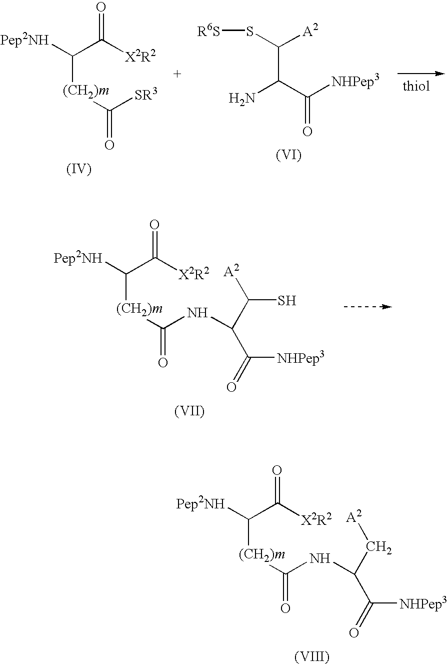

wherein X² is O or NH; R² is an alkyl group, an alkenyl group, an aryl group, an alkyl-substituted aryl group, an aryl-substituted alkyl group, an amino acid, or a peptide, optionally including one or more protecting groups on a nitrogen, oxygen, or sulfur substitutent thereof; R³ and R⁶ are each independently an alkyl group, an alkenyl group, an aryl group, an alkyl-substituted aryl group, or an aryl-substituted alkyl group; Pep² and Pep³ are each independently an amino acid or a peptide, which optionally includes one or more protecting groups on a nitrogen, oxygen, or sulfur substitutent thereof; A² is phenyl, 4-hydroxyphenyl, or 3-indolyl; and m is 1 or 2. In a preferred embodiment, R³ and R⁶ are each independently C₁-C₄ alkyl (e.g., ethyl) or benzyl; preferably R³ and R⁶ are each ethyl. Preferably, the thiol is a 2-mercaptoethanesulfonic acid salt. In some preferred embodiments, X² is NH, and R² is a peptide or an amino acid, optionally comprising one or more protecting groups on a nitrogen, oxygen, or sulfur substitutent thereof. This method extends the native chemical ligation methodology from the known ligation at cysteine residues to the novel ligation at phenylalanyl, trosyl and tryptophanyl residues.

The preliminary results outlined for phenylalanine in Scheme 17 are extended to a practical method compatible with the standard Fmoc-based peptide synthesis protocol.^{135, 212-214} Thus, amine 8 is converted to its Fmoc-protected form and the methyl ester saponified under conditions known not to cause epimerization of the α-center,²¹⁴⁻²¹⁶ giving the building block 96 (Scheme 37). This amino-acid is then used to cap the N-terminal end of peptide segments prepared by the Fmoc-method, either in solution for short di- and tripeptides, by manual solid phase peptide synthesis, or on the synthesizers available in the campus peptide synthesis facility (Protein Research Laboratory, Research Center), with final removal of the Fmoc group with piperidine (Scheme 37). As in the model studies these peptides carrying N-terminal modified phenylalanine groups serve as lynchpins in the native chemical ligation to a second C-terminal thiolester. Finally, the thiol group is removed by established methods,⁶⁴ again as demonstrated in the preliminary results, to afford the native peptide with its phenylalanine residue.

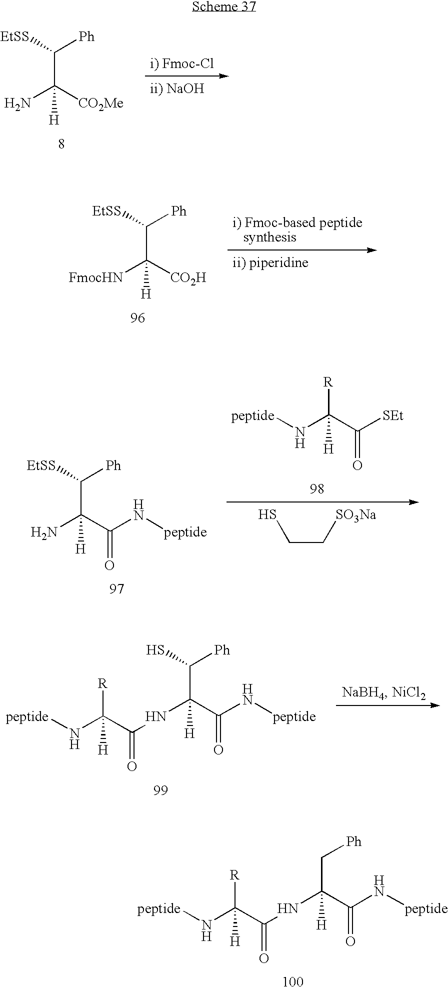

In addition to the β-mercaptophenylalanine ligation, analogous chemistry is developed for β-mercapto-tyrosine and tryptophan derivatives to determine their application in native chemical ligation (Scheme 38).

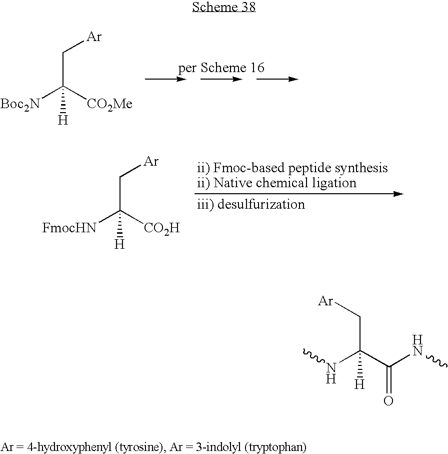

To evaluate the ligation protocols of the invention, the chemistry of Dawson and his LYRAX (SEQ ID NO: 5) and CRANK (SEQ ID NO: 4) sequences used to previously probe the scope of native chemical ligation at cysteine¹⁸⁵ is adapted and employed in challenging the present cysteine functionalization chemistries. Thus, the Dawson CRANK pentapeptide (SEQ ID NO: 5) is modified to ZRANK (SEQ ID NO: 10) in which Z is either of the side chain functionalized phenylalanine, tyrosine, or tryptophan residues, or the thioaspartates described above, and this system is used for coupling to LYRAX (SEQ ID NO: 5). In this manner, decapeptides LYRAXZRANK (SEQ ID NO: 11) are obtained, and through systematic variation of X the scope of new ligation chemistries is ascertained. As discussed previously, all peptides segments for ligation are prepared on the 0.25 mmol scale employed by Dawson using manual solid phase peptide synthesis, and all peptides are purified by reverse phase HPLC with analysis by NMR and ESI and/or MALDI-TOF mass spectrometry as appropriate.

Finally, attention is directed to the Aβ(1-42) β-amyloid peptide, which, together with the Aβ(1-40) segment, is the primary constituent of the neurotoxic oligomers whose presence has been linked to the progression of Alzheimer's Disease.²²⁰⁻²²⁷ In addition to the biological relevance and the need for synthetic Aβ(1-42) for Alzheimer's research, this peptide has become a test bed for peptide synthesis methodologies because of the widely-appreciated challenges in its synthesis, particularly aggregation. For example, since the solution phase synthesis of Aβ(1-42) by Fmoc-based methods with HATU mediated couplings by the Fukuda group,²²⁵ numerous approaches have been described including polymer-supported syntheses on polystyrene,^{228, 229} Tentagel,²³⁰ PEG-polystyrene,^{230, 231} Pepsyn K,²³² and a cross-linked PEG-based resin,²³³ with varying degrees of success. Pertinently, a solution phase segment coupling synthesis has also been conducted using a Boc-protection strategy and HOBT/carbodiimide coupling, but in the rather unusual solvent mixture of phenol and chloroform.²³⁴ One example of the type of retrosynthetic analysis of the Aβ(1-42) β-amyloid peptide 110 (SEQ ID NO: 12) to be pursued is given in Scheme 39. This analysis takes advantage of the conveniently spaced aspartic acid, phenylalanine, and asparagines residues to dissect the target into four manageable segments 111-114 (111, SEQ ID NO: 13; 112, SEQ ID NO: 14; 113, SEQ ID NO: 15; and 114, (SEQ ID NO: 16).

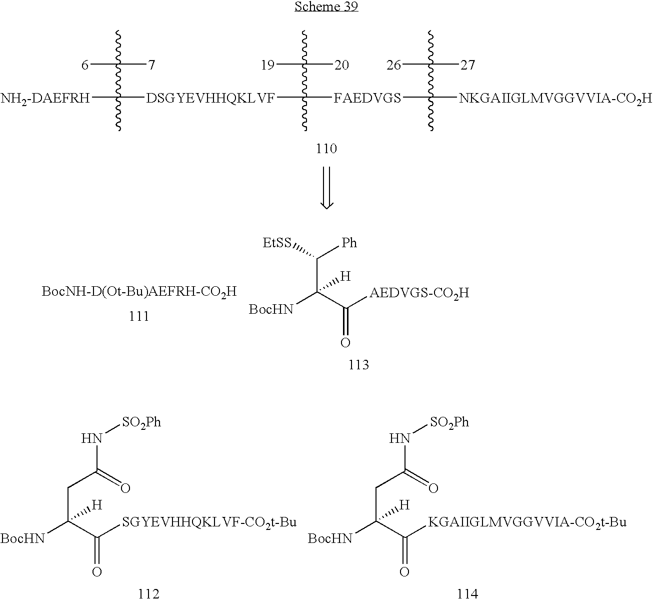

Consideration was given to adapting the thiazolidine protection scheme,^{235, 236} employed by Kent in his convergent syntheses of crambin and other peptides,^{58, 59} to the N-terminal β-mercaptophenylalanine in fragment 113 (SEQ ID NO: 15), but the problems of combining native chemical ligation with segment coupling are better solved by incorporating segments 112 (SEQ ID NO: 14) and 113 (SEQ ID NO: 15) as the C-terminal acids. All four segments are prepared by manual Boc solid phase peptide synthesis protocols, based on the procedure of Kent,²³⁷ as adopted by Janda in his methionine sulfoxide-modified linear synthesis of the complete sequence,²²⁹ and as used by Nishiuchi in his segment condensation approach.²³⁴ Activation of 111 (SEQ ID NO: 13) with HATU in the presence of a thiol affords the corresponding thioester 115 (SEQ ID NO: 17) ready for native chemical ligation. Conversion of 112 (SEQ ID NO: 14) to the thioaspartate is achieved with iodoacetonitrile and NaSH, as set out in Scheme 36, following which the Boc and t-Bu protecting groups are removed with TFA, giving 116 (SEQ ID NO: 18). After native ligation chemical of 115 (SEQ ID NO: 17) with 116 (SEQ ID NO: 18) the thioaspartate side chain of the product 117 (SEQ ID NO: 19) is activated with iodoacetamide and converted to the protected aspartate 118 (SEQ ID NO: 20) with t-butanol (Scheme 40).

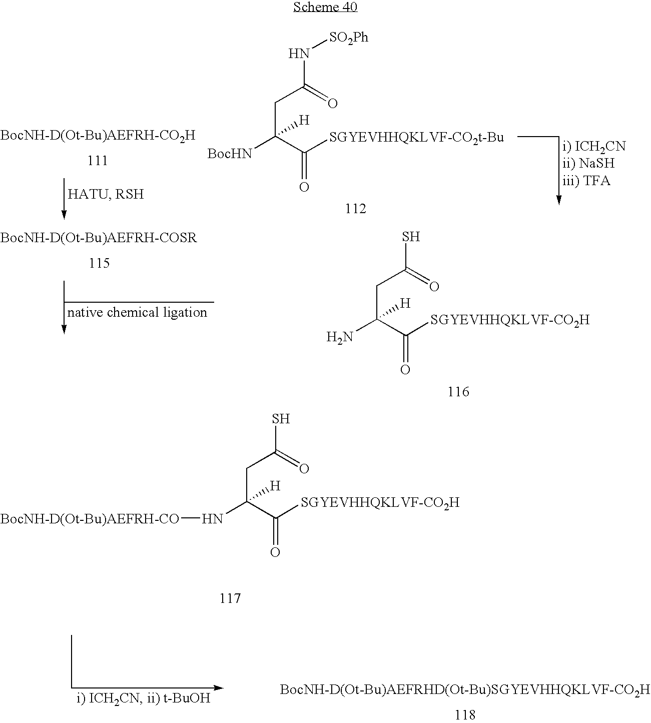

Following activation of 118 (SEQ ID NO: 20) with HATU and thiol, removal of the Boc group from 113 (SEQ ID NO: 15) with TFA permits a second native chemical ligation step (Scheme 41). This is followed up with removal of the thiol group from the phenylalanine side chain by the conditions already established in the preliminary work (Scheme 17).

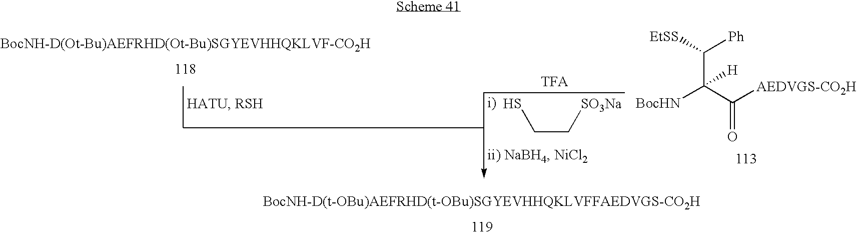

The final native chemical ligation step involves activation of 119 (SEQ ID NO: 21) in the form of its thioester 120 (SEQ ID NO: 22) and conversion of segment 114 (SEQ ID NO: 16) to the thioaspartate 121 (SEQ ID NO: 23) along the established lines. After this final coupling, the remaining thioaspartate in 122 (SEQ ID NO: 24) is converted to the protected asparagine side chain with iodoacetamide and then xanthyl amine, and the complete protected sequence 123 (SEQ ID NO: 25) purified by reverse phase HPLC before the final treatment with HF, which removes all Boc groups, t-butyl esters, and the xanthyl group (Scheme 42) and affords the target 110 (SEQ ID NO: 12).

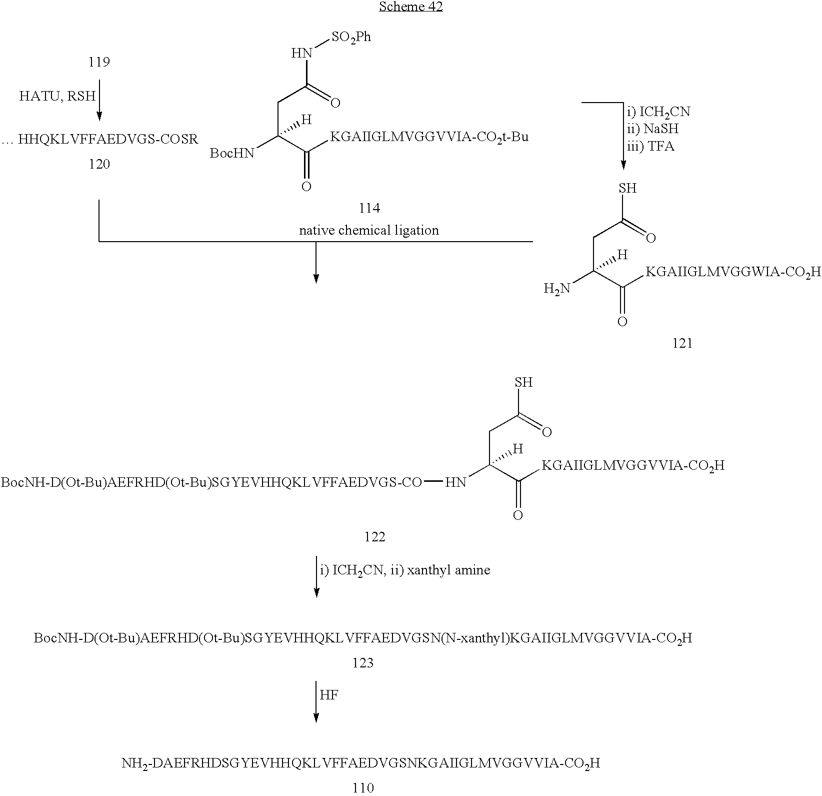

All three native chemical ligation steps are conducted in DMF solution and the products are purified by reverse phase HPLC, with confirmation of structure by ESI and/or MALDI-TOF mass spectrometry.

### Functionalization of Thiols by Sigmatropic Rearrangement

Preliminary work relating to the permanent ligation of thiols by the desulfurative rearrangement of allylic disulfides has been published in Organic Letters.²³⁸ A total of thirteen examples were presented illustrating the range of thiols that may be functionalized in this manner, and of the groups to which they may be attached. For illustrative purposes, a single example is given here in which an aliphatic chain was conjugated to glutathione in the absence of any protecting groups in a mixture of tris buffer and acetonitrile. The entire sequence was conducted in one pot without isolation of the disulfide intermediate, and the product was formed as a pure trans-isomer.

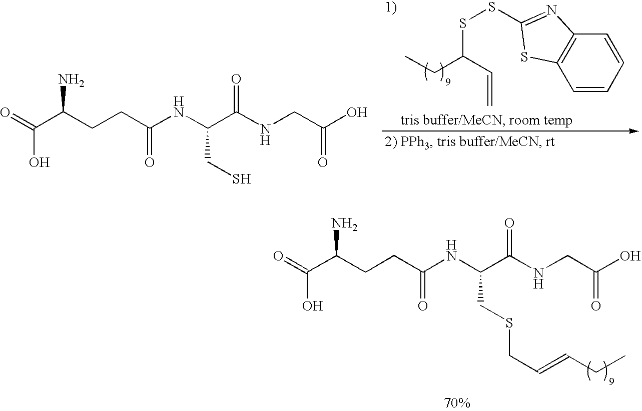

Subsequently, according to the present invention, the chemistry has been applied to a commercial decapeptide (SEQ ID NO: 26), again in tris buffer at room temperature, and in the complete absence of protecting groups to form the allylic sulfide derivative SEQ ID NO: 27 in about 70% yield, as shown below.

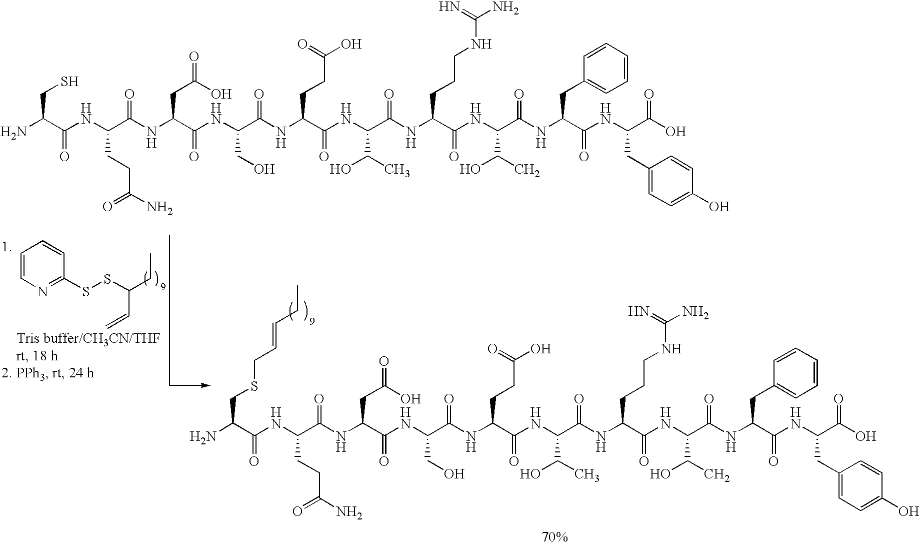

Proof of principle has been established for the conjugation of a sugar to a peptide in aqueous solution in the complete absence of protecting groups. This reaction can be applied to higher sugars and larger peptides.

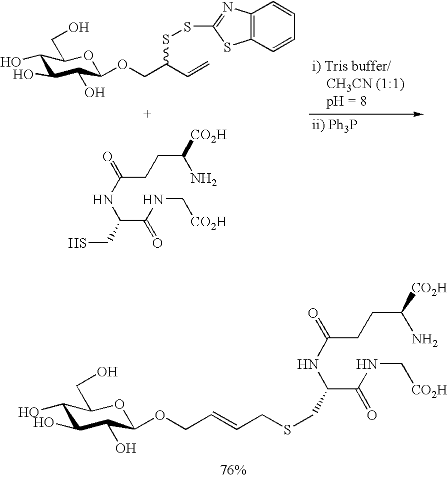

Ongoing studies of the desulfurative rearrangement have also uncovered the fact that the phosphine is not an absolute requirement. Thus, as noted in the preliminary communication,²³⁸ when the allylic disulfide is dissolved in CDCl₃ that has not been filtered through basic alumina the desulfurative rearrangement takes place spontaneously at room temperature over a period of several hours. In some instances the desulfurative rearrangement was also observed to take place on silica gel chromatography.²³⁸ While these observations are only preliminary, and have yet to be followed up on, they certainly indicate that phosphine-free conditions can be found for this ligation.

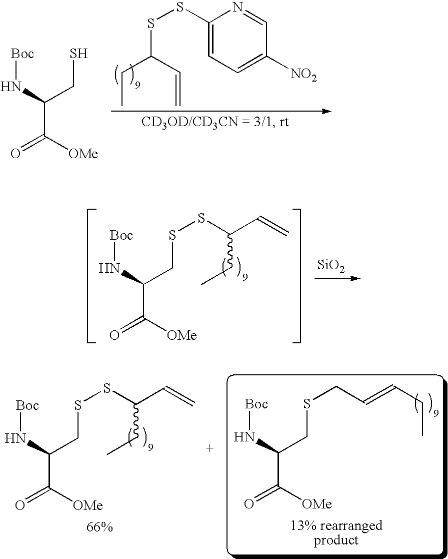

A carbohydrate-derived diazoamide has been prepared and conjugated to an S-allyl cysteine derivative in 54% yield using the Doyle-Kirmse reaction. The mass balance in this reaction was comprised of the dimerized carbenoid and unreacted cysteine derivative, rather than of any NH insertion products. The choice of the diazo amide, rather than the more common diazo ester, was deliberate. The highly preferred trans-geometry of the amide linkage serves to protect against self reaction of the carbenoid moiety with the sugar, i.e., it prevents biting back. The amide is also a better mimic of the N-glycosyl asparagine linkage that an ester.

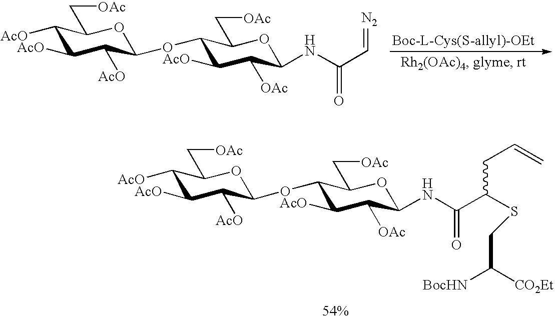

### Asparagine Glycosylation.

In a proof of principle, the Tomkinson variant on the Fukuyama sulfonamide coupling protocol was successfully applied to a glycosylated sulfonamide.

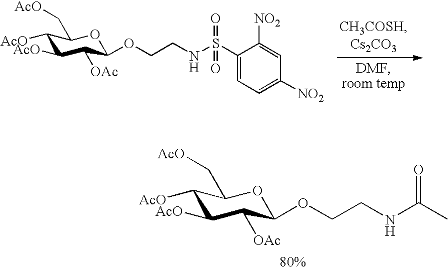

A pentapeptide containing the β-ethyldithio phenylalaninyl group (XRANK, SEQ ID NO: 28) was successfully prepared on 100 mg scale by solid phase peptide synthesis in the Protein Research Laboratory of the UIC Research Resources Center. A second pentapeptide thioester (LYRAM-SBn, SEQ ID NO: 29) was similarly assembled and the two were combined by native chemical ligation method of the invention to afford decapeptide LYRAMXRANK, SEQ ID NO: 30). These two peptides were selected to illustrate the broad functional group compatibility of the chemistry. The product (SEQ ID NO: 30) was isolated by reverse phase HPLC and its structure established by mass spectrometric methods. The desulfurization should not be a problem on the basis of the results reported in Scheme 17 of the proposal.

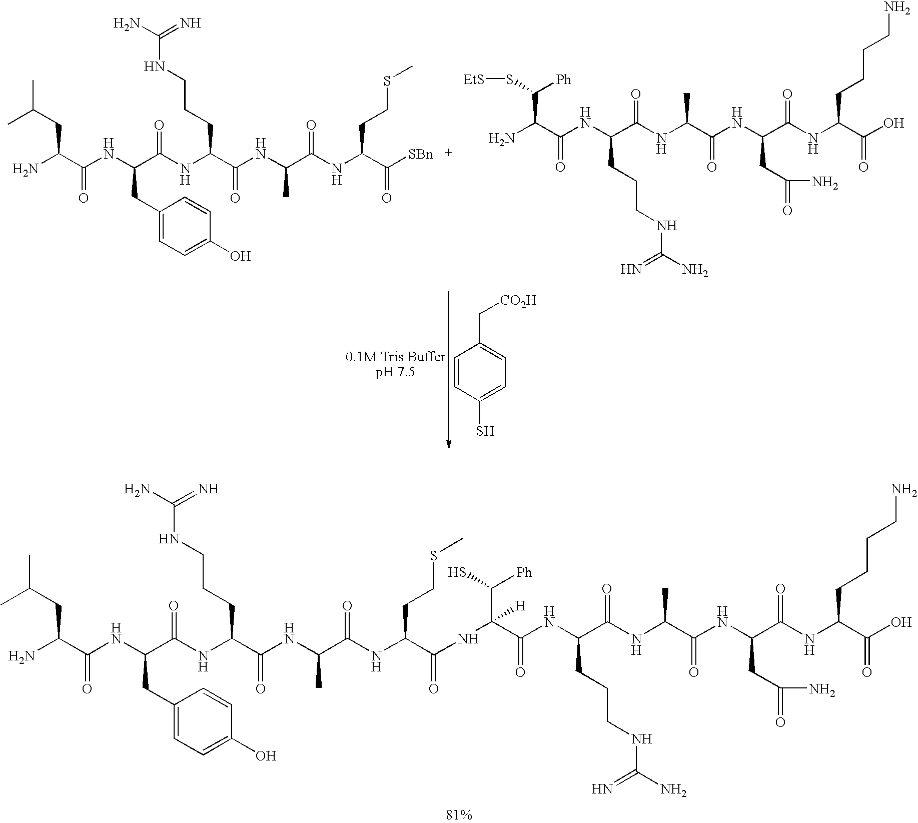

In summary, the present invention has established the following: i) That the desulfurative ligation is applicable to peptides in aqueous solution in the absence of protecting groups, and is compatible with most of the more reactive amino acid side-chains. ii) The desulfurative ligation is compatible with the glycosylation of a peptide in aqueous solution in the absence of protecting groups. iii) Carbohydrate-based diazo amides may be readily prepared, do not bite back on themselves, and can be employed in the Doyle-Kirmse reaction to derivatize peptides previously functionalized by the present new ligation chemistry. iv) The Tomkinson modification of the Fukuyama sulfonamide chemistry is applicable to the formation of glycoconjugates. v) The UIC Protein Research Laboratory is capable and willing to prepare precursors for native chemical ligation. vi) The functionalized phenylalanine can be incorporated into N-terminal peptides by solid phase peptide synthesis. vii) That the native chemical ligation functions, at least for the synthesis of an octapeptide selected deliberately to include the most difficult amino acid side chains.

### CITED REFERENCES

1. Paulson, J. C.; Blixt, O.; Collins, B. E. Sweet spots in functional glycomics Nature Chemical Biology 2006, 2, 238-248.
2. Kiessling, L. L.; Gestwicki, J. E.; Strong, L. E. Synthetic multivalent ligands as probes of signal transduction Angew. Chem. Int. Ed. 2006, 45, 2348-2368.
3. Pratt, M. R.; Bertozzi, C. R. Synthetic glycopeptides and glycoproteins as tools for biology Chem. Soc. Rev 2005, 34, 58-68.
4. Dwek, R. A.; Butters, T. D. Glycobiology—Understanding the Language and Meaning of Carbohydrates Chem. Rev. 2002, 102, 283-284.
5. Liu, L.; Bennett, C. S.; Wong, C.-H. Advances in glycoprotein synthesis Chem. Commun. 2006, 21-33.
6. Wong, C.-H. Protein glycosylation: New Challenges and Opportunities J. Org. Chem. 2005, 70, 4219-4225.
7. Principles of Proteomics, Twyman, R. M., 2003, BIOS Scientific, Oxford, pp 350.
8. The proteomics protocols handbook, Eds. Walker, J. M., 2005, Humana Press, Totowa, pp 988.
9. Proteomics of disease, Eds. Celius, J. E.; Korc, M., 2005, American Society for Biochem. and Mol. Biol., Bethesda, pp 258.
10. Biomedical Applications of Proteomics, Eds. Sanchez, J.-C.; Corthals, G. L.; Hochstrasser, D. F., 2004, Wiley-VCH, Weinheim, pp 435.
11. Davis, B. G. Synthesis of Glycoproteins Chem. Rev. 2002, 102, 579-601.
12. Pachamuthu, K.; Schmidt, R. R. Synthetic routes to thiooligosaccharides and thioglycopeptides Chem. Rev. 2006, 106, 160-187.
13. Neoglycoconjugates: Preparation and Applications, Eds. Lee, Y. C.; Lee, R. T., 1994, Academic Press, San Diego.
14. Roy, R. In Carbohydrate Chemistry, Eds. Boons, G.-J., 1998, Blackie Academic and Professional, London, 243-321.
15. Köhn, M.; Breinbauer, R. The Staudinger Ligation—A Gift to Chemical Biology Angew. Chem. Int. Ed. 2004, 43, 3106-3116.
16. Breinbauer, R.; Köhn, M. Azide-Alkyne Coupling: A Powerful reaction for Bioconjugate Chemistry Chem BioChem 2003, 4, 1147-1149.
17. Van Swieten, P. F.; Leeuwenburgh, M. F.; Kessler, B. M.; Overkleeft, H. S. Bioorthogonal organic chemistry in living cells: novel strategies for labeling biomolecules Org. Biomol. Chem. 2005, 3, 20-27.
18. Peri, F.; Nicotra, F. Chemoselective ligation in glycochemistry Chem Comm 2004, 623-627.
19. Langenhan, J. M.; Thorson, J. S. Recent carbohydrate-based chemoselective ligation applications Current Organic Synthesis 2005, 2, 59-81.
20. Kolb, H. C.; Finn, M. G.; Sharpless, K. B. Click Chemistry: Diverse Chemical Function from a Few Good Reactions Angew. Chem. Int. Ed. 2001, 40, 2004-2021.
21. Kaneshiro, C. M.; Michael, K. A convergent synthesis of N-glycopeptides Angew. Chem. Int. Ed. 2006, 45, 1077-1081.
22. Ragupathi, G.; Koide, F.; Livingston, P. O.; Cho, Y. S.; Endo, A.; Wan, Q.; Spassova, M. K.; Keding, S. J.; Allen, J.; Ouerfelli, O.; Wilson, R. M.; Danishefsky, S. J. Preparation and evaluation of unimolecular pentavalent and hexavalent antigenic constructs targeting prostate and breast cancer: a synthetic route to anticancer vaccine candidates J. Am. Chem. Soc. 2006, 128, 2715-2725.
23. Galonic, D. P.; Ide, N. D.; Van Der Donk, W. A.; Gin, D. Y. Aziridine-2-carboxylic Acid-Containing Peptides: Application to Solution- and Solid-Phase Convergent Site-Selective Peptide Modification J. Am. Chem. Soc. 2005, 127, 7359-7369.
24. Thayer, D. A.; Yu, H. N.; Galan, M. C.; Wong, C.-H. A General Strategy toward S-Linked Glycopeptides Angew. Chem. Int. Ed. 2005, 44, 4596-4599.
25. Opatz, T.; Kallus, C.; Wunberg, T.; Kunz, H. Combinatorial synthesis of amino acid- and peptide-carbohydrate conjugates on solid phase Tetrahedron 2004, 60, 8613-8626.
26. Zhu, X. M.; Pachamuthu, K.; Schmidt, R. R. Synthesis of novel S-neoglycopeptides from glycosylthiomethyl derivatives Org. Lett. 2004, 6, 1083-1085.
27. Knapp, S.; Myers, D. S. Synthesis of a-GalNAc Thioconjugates from an a-GalNAc Mercaptan J. Org. Chem. 2002, 67, 2995-2999.
28. Zhu, X. M.; Schmidt, R. R. Efficient Synthesis of S-Linked Glycopeptides in Aqueous Solution by a Convergent Strategy Chem. Eur. J. 2004, 10, 875-887.
29. Pachamuthu, K.; Zhu, X.; Schmidt, R. R. Reversed approach to S-farnesylation and S-palmitoylation: application to an efficient synthesis of the C-terminus of lipidated humanN-RAS hexapeptide J. Org. Chem. 2005, 70, 3720-3723.
30. Jobron, L.; Hummel, G. Solid-Phase Synthesis of New S-Glycoamino Acid Building Blocks Org. Lett. 2000, 2, 2265-2267.
31. Cohen, S. B.; Halcomb, R. L. Synthesis of S-Linked Glycosyl Amino Acids in Aqueous Solution with Unprotected Carbohydrates Org. Lett. 2001, 3, 405-407.
32. Palomo, J. M.; Lumbierres, M.; Waldmann, H. Efficient Solid-Phase Lipopeptide Synthesis Employing the Ellman Sulfonamide Linker Angew. Chem. Int. Ed. 2006, 45, 477-481.
33. Tilley, D. S.; Francis, M. B. Tyrosine-selective Protein Alkylation using p-allylpalladium complexes J. Am. Chem. Soc. 2006, 128, 1080-1081.
34. Avenoza, A.; Busto, J. H.; Jiménez-Osés, G.; Peregrina, J. M. Enantioselective synthesis of S-linked glycosyl-b^{2,2}-amino acid derivatives by S_{N}2 reaction with hindered sulfamidates Synthesis 2006, 641-643.
35. Ichikawa, Y.; Matsukawa, Y.; Isobe, M. Synthesis of Urea-Tethered Neoglycoconjugates and Pseudooligosaccharides in Water J. Am. Chem. Soc. 2006, 128, 3934-3938.
36. Prescher, J. A.; Dube, D. H.; Bertozzi, C. R. Chemical remodelling of cell surfaces in living animals Nature 2004, 430, 873-877.
37. Hang, H. C.; Yu, C.; Pratt, M. R.; Bertozzi, C. R. Probing glycosyl transferase activities with the Staudinger ligation J. Am. Chem. Soc. 2004, 126, 6-7.
38. Lemieux, G. A.; De Graffenreid, C. L.; Bertozzi, C. R. A fluorogenic dye activated by the Staudinger ligation J. Am. Chem. Soc. 2003, 125, 4708-4709.
39. Saxon, E.; Luchansky, S. J.; Hang, H. C.; Yu, C.; Lee, S. C.; Bertozzi, C. R. Investigating cellular metabolism of synthetic azidosugars with the Staudinger ligation J. Am. Chem. Soc. 2002, 124, 14893-14902.
40. Saxon, E.; Bertozzi, C. R. Cell surface engineering by a modified Staudinger reaction Science 2000, 287, 2007-2010.
41. Watzke, A.; Köhn, M.; Gutierrez-Rodriguez, M.; Wacker, R.; Schröder, H.; Breinbauer, R.; Kuhlmann, J.; Alexandrov, K.; Niemeyer, C. M.; Goody, R. S.; Waldmann, H. Site-selective protein immobilization by Staudinger Ligation Angew. Chem. Int. Ed. 2006, 45, 1408-1412.
42. Hang, H. C.; Bertozzi, C. R. Chemoselective approaches to glycoprotein assembly Acc. Chem. Res. 2001, 34, 727-736.
43. Gololobov, Y. G.; Kasukhin, L. F. Recent Advances in the Staudinger Reaction Tetrahedron 1992, 48, 1353-1406.
44. Lin, F. L.; Hoyt, H. M.; Van Halbeek, H.; Bergman, R. G.; Bertozzi, C. R. Mechanistic Investigation of the Staudinger Ligation J. Am. Chem. Soc. 2005, 127, 2686-2695.
45. Saxon, E.; Armstrong, J. I.; Bertozzi, C. R. A “Traceless” Staudinger Ligation for the Chemoselective Synthesis of Amide Bonds Org. Lett. 2000, 2, 2141-2143.
46. Dudkin, V. Y.; Crich, D. A Short Synthesis of the Trisaccharide Building Block of the N-Linked Glycans Tetrahedron Lett. 2003, 44, 1787-1789.
47. Rakotomanomana, N.; Lacombe, J.-M.; Pavia, A. Reduction-acetylation selective des azido-sucres par le melange acide thioacétique-thioacétate de potassium Carbohydr. Res. 1990, 197, 318-323.
48. Shangguan, N.; Katukojvala, S.; Greenberg, R.; Williams, L. J. The Reaction of Thio Acids with Azides: A New Mechanism and New Synthetic Applications J. Am. Chem. Soc. 2003, 125, 7754-7755.
49. Barlett, K. N.; Kolakowski, R. V.; Katukojvala, S.; Williams, L. J. Thio Acid/Azide Amidation: An Improved Route to N-Acyl Sulfonamides Org. Lett. 2006, 8, 823-826.
50. Merkx, R.; Brouwer, A. R.; Rijkers, D. T. S.; Liskamp, R. M. J. Highly Efficient Coupling of b-Substituted Aminoethane Sulfonyl Azides with Thio Acids, Toward a New Chemical Ligation Reaction Org. Lett. 2005, 7, 1125-1128.
51. Knapp, S.; Darout, E. New Reactions of Selenocarboxylates Org. Lett. 2005, 7, 203-206.
52. Kolakowski, R. V.; Shangguan, N.; Williams, L. J. Thioamides via thiatriazolines Tetrahedron Lett. 2006, 47, 1163-1166.
53. Dawson, P. E.; Muir, T. W.; Clark-Lewis, I.; Kent, S. B. H. Synthesis of proteins by native chemical ligation Science 1994, 266, 776-779.
54. Kochendoerfer, G. G.; Kent, S. B. H. Chemical Protein Synthesis Curr. Opinion. Chem. Biol. 1999, 3, 665-671.
55. Dawson, P. E.; Kent, S. B. H. Synthesis of native proteins by chemical ligation Ann. Rev. Biochem. 2000, 69, 923-960.
56. Coltart, D. M. Peptide Segment Coupling by Prior Ligation and Proximity-Induced Intramolecular Acyl Transfer Tetrahedron 2000, 56, 3449-3491.
57. Yeo, D. S. Y.; Srinivasan, R.; Chen, G. Y. J.; Yao, S. Q. Expanded Utility of the Native Chemical Ligation Reaction Chem. Eur. J. 2004, 10, 4664-4672.
58. Bang, D.; Kent, S. B. H. His₆ tag-assisted chemical protein synthesis Proc. Natl. Acad. Sci., U.S.A. 2005, 102, 5014-5019.
59. Bang, D.; Kent, S. B. H. A one-pot synthesis of crambin Angew. Chem. Int. Ed. 2004, 43, 2534-2538.
60. Nilsson, B. L.; Soellner, M. B.; Raines, R. T. Chemical synthesis of proteins 2005, 34, 91-118.
61. Dose, C.; Seitz, O. Convergent Synthesis of Peptide Nucleic Acids by Native Chemical Ligation Org. Lett. 2005, 7, 4365-4368.
62. Johnson, E. C. B.; Durek, T.; Kent, S. B. H. Total Chemical Synthesis, Folding, and Assay of a Small Protein on a Water-Compatible Solid Support Angew. Chem. Int. Ed. 2006, 45, 3283-3287.
63. Johnson, E. C. B.; Kent, S. B. H. Insights into the Mechanism and Catalysis of the Native Chemical Ligation Reaction J. Am. Chem. Soc. 2006, 128, 6640-6646.
64. Yan, L. Z.; Dawson, P. E. Synthesis of peptides and proteins without cysteine residues by native chemical ligation combined with desulfurization J. Am. Chem. Soc. 2001, 123, 526-533.
65. Hondal, R. J.; Nilsson, B. L.; Raines, R. T. Selenocysteine in Native Chemical Ligation and Expressed Protein Ligation J. Am. Chem. Soc. 2001, 123, 5140-5141.
66. Canne, L. E.; Bark, S. J.; Kent, S. B. H. Extending the applicability of native chemical ligation J. Am. Chem. Soc. 1996, 118, 5891-5896.
67. Botti, P.; Carrasco, M. R.; Kent, S. B. H. Native chemical ligation using removable N^{a}-(1-phenyl-2-mercaptoethyl) auxiliaries Tetrahedron Lett. 2001, 42, 1831-1833.
68. Clive, D. L. J.; Hisaindee, S.; Coltart, D. M. Derivatized Amino acids reelvant to native peptide synthesis by chemical ligation and acyl transfer J. Org. Chem. 2003, 68, 9247-9254.
69. Tchertchian, S.; Hartley, O.; Botti, P. Synthesis of Na-(i-Phenyl-2-mercaptoethyl) Amino Acids, New Building blocks for Ligation and Cyclization at Non-Cysteine Sites: Scope and Limitations in Peptide Synthesis J. Org. Chem. 2004, 69, 9208-9214.
70. Offer, J.; Dawson, P. E. N^{a}-2-Mercaptobenzylamine-Assisted Chemical Ligation Org. Lett. 2000, 2, 23-26.
71. Lu, Y.-A.; Tam, J. P. Peptide Ligation by a Reversible and Reusable C-Terminal Thiol Handle Org. Lett. 2005, 7, 5003-5006.
72. Macmillan, D.; Anderson, D. W. Rapid synthesis of acyl transfer auxiliaries for cysteine-free native glycopeptide ligation Org. Lett. 2004, 6, 4659-4662.
73. Ueda, S.; Fujita, M.; Tamamura, H.; Fujii, N.; Otaka, A. Photolabile protection for one-pot sequential native chemical ligation 2005, 6, 1983-1986.
74. Marinzi, C.; Offer, J.; Longhi, R.; Dawson, P. E. An o-nitrobenzyl scaffold for peptide ligation: synthesis and applications 2004, 12, 2749-2757.
75. Brik, A.; Yang, Y.-Y.; Ficht, S.; Wong, C.-H. Sugar-assisted Glycopeptide Ligation J. Am. Chem. Soc. 2006, 128, 5626-5627.
76. Nilsson, B. L.; Kiessling, L. L.; Raines, R. T. High-yielding Staudinger ligation of a phosphinothioester and azide to form a peptide Org. Lett. 2001, 3, 9-12.
77. Nilsson, B. L.; Hondal, R. J.; Soellner, M. B.; Raines, R. T. Protein assembly by orthogonal chemical ligation methods J. Am. Chem. Soc. 2003, 125, 5268-5269.
78. He, Y.; Hinklin, R. J.; Chang, J.; Kiessling, L. Stereoselective N-Glycosylation by Staudinger Ligation Org. Lett. 2004, 6, 4479-4482.
79. Grandjean, C.; Boutonnier, A.; Guerreiro, C.; Fournier, J.-M.; Mulard, L. A. On the Preparation of Carbohydrate-Protein Conjugates Using the Traceless Staudinger Ligation J. Org. Chem. 2005, 70, 7123-7132.
80. Doores, K. J.; Mimura, Y.; Dwek, R. A.; Rudd, P. M.; Elliott, T.; Davis, B. G. Direct deprotected glycosyl-asparagine ligation Chem. Commun. 2006, 1401-1403.
81. Kenyon, G. L.; Bruice, T. W. Novel Sulfhydryl Reagents Methods Enzym. 1977, 47, 407-430.
82. Wynn, R.; Richards, F. M. Chemical modification of protein thiols: formation of mixed disulfides Methods Enzym. 1995, 251, 351-356.
83. Faulstich, H.; Heintz, D. Reversible introduction of thiol compounds into proteins by use of activated mixed disulfides Methods Enzym. 1995, 251, 357-366.
84. Chemical Reagents for Protein Modification, Lundblad, R. L., 2005, CRC Press, Boca Raton, pp 339.
85. Bioconjugate Techniques, Hermanson, G. T., 1996, Academic Press, San Diego, pp 785.
86. Macindoe, W. M.; Van Oijen, A. H.; Boons, G.-J. A unique and highly facile method for synthesizing disulfide linked neoglycoconjugates: a new approach for remodelling of peptides and proteins Chem. Commun. 1998, 847-848.
87. Gamblin, D. P.; Garnier, S. J.; Ward, N. J.; Oldham, A. J.; Fairbanks, A. J.; Davis, B. G. Glycosyl phenylthiosulfonates (Glyco-PTS): novel reagents for glycoprotein synthesis Org. Biomol. Chem. 2003, 1, 3642-3644.
88. Gamblin, D. P.; Garnier, P.; Van Kasteren, S.; Oldham, N. J.; Fairbanks, A. J.; Davis, B. G. Glyco-SeS Selenenylsulfide-Mediated Protein Glycoconjugation-A New Strategy in Postranslational Modification Angew. Chem. Int. Ed. 2004, 43, 828-833.
89. Ludolph, B.; Eisele, F.; Waldmann, H. Solid-Phase Synthesis of Lipidated Peptides J. Am. Chem. Soc. 2002, 124, 5954-5955.
90. Zhu, Y.; Van Der Donk, W. A. Convergent Synthesis of Peptide Conjugates Using Dehydroalanines for Chemoselective Ligations Org. Lett. 2001, 3, 1189-1192.
91. Cohen, S. B.; Halcomb, R. L. Application of Serine- and Threonine-Derived Cyclic Sulfamidates for the Preparation of S-Linked Glycosyl Amino Acids in Solution- and Solid-Phase Peptide Synthesis J. Am. Chem. Soc. 2002, 124, 2534-2543.
92. Galonic, D. P.; Van Der Donk, W.; Gin, D. Y. Site-Selective Conjugation of Thiols with Aziridine-2-carboxylic acid-containing peptides J. Am. Chem. Soc. 2004, 126, 12712-12713.
93. Durek, T.; Alexandrov, K.; Goody, R. S.; Hildebrand, A.; Heinemann, I.; Waldmann, H. Synthesis of Fluorescently Labeled Mono- and Diprenylated Rab7 GTPase J. Am. Chem. Soc. 2004, 126, 16368-16378.
94. Kragol, G.; Lumbierres, M.; Palomo, J. M.; Waldmann, H. Solid Phase Synthesis of Lipidated Peptides Angew. Chem. Int. Ed. 2004, 43, 5839-5842.
95. Organic selenium compounds: their chemistry and biology, Eds. Klayman, D. L.; Gunther, W. H. H., 1973, Wiley Interscience, New York, pp 1188.
96. Price, T. S.; Jones, L. M. The benzyl and nitrobenzyl selenosulfates and the benzyl and nitrobenzyl diselenides J. Chem. Soc. 1909, 95, 1729.
97. Günther, W. H. H.; Mautner, H. G. Analogs of parasympathetic neuroeffectors. Acetylcholine, selenocholine, and related compounds J. Med. Chem. 1964, 7, 229-232.
98. Patarasakulchai, N.; Southwell-Keely, P. T. Comparative Reactivities of Selenosulfates and Selenenylthiosulfates with Thiols Phosphorus and Sulfur 1985, 22, 277-282.
99. Höfle, G.; Baldwin, J. E. Thiosulfoxides. The Intermediates in Rearrangement and Reduction of Allylic Disulfides J. Am. Chem. Soc. 1971, 93, 6307-6308.
100. Crich, D.; Krishnamurthy, V.; Hutton, T. K. Allylic Selenosulfide Rearrangement: A Method for Chemical Ligation to Cysteine and Other Thiols J. Am. Chem. Soc. 2006, 128, 2544-2545.
101. Harano, K.; Taguchi, T. Rearrangement and trans-elimination contrary to the Chugaev reaction rule. XIII. Solvent effect on the rearrangement reaction of allylic xanthates and a correction of the former report Chem. Pharm. Bull. 1975, 23, 467-472.
102. Harano, K.; Taguchi, T. Stereochemistry. XLVII. Rearrangement and trans-elimination contrary to the Chugaev reaction rule. VIII. Thermal rearrangement of 2-alkenyl S-alkyl xanthate Chem. Pharm. Bull. 1972, 20, 2348-2356.
103. Harano, K.; Taguchi, T. Stereochemistry. XLVIII. Rearrangement and trans-elimination contrary to the Chugaev reaction rule. IX. Thermal rearrangement of 2-cycloalkenyl S-alkyl xanthate Chem. Pharm. Bull. 1972, 20, 2357-2365.
104. Nakai, T.; Mimura, T.; Ari-Izumi, A. Dithiocarbamates in organic synthesis. Part VI. A new method for the bishomologation of carbonyl compounds to a,b-unsaturated aldehydes via the [3,3]-sigmatropic rearrangement of allylic thionocarbamates Tetrahedron Lett. 1977, 2425-2428.
105. Ueno, Y.; Sano, H.; Okawara, M. Synthetic reactions using organotin and sulfur compounds. Part 4. Deoxygenation of allylic alcohols to terminal olefins via stannylation/destannylation Tetrahedron Lett. 1980, 21, 1767-1770.
106. Eto, M.; Tajiri, 0.; H., N.; K., H. Correlation of Thione-to-thiol Rearrangement Rates of Xanthates with Solvent Scales. Analysis of the Reaction Behavior by the Kamlet-Taft Parameters, p*, a and b Tetrahedron 1998, 54, 8009-8014.
107. Evans, D. A.; Andrews, G. C. Allylic Sulfoxides: Useful Intermediates in Organic Synthesis Acc. Chem. Res. 1974, 7, 147-155.
108. Nakai, T.; Mikami, K. In Org. React., Eds. Paquette, L. A., 1994, John Wiley & Sons, New York, 105-209.
109. Nakai, T.; Mikami, K. [2,3]-Wittig Sigmatropic Rearrangements in Organic Synthesis Chem. Rev. 1986, 86, 885-902.
110. Mikami, K.; Nakai, T. Acyclic Stereocontrol via [2,3]-Wittig Sigmatropic Rearrangement Synthesis 1991, 594-604.
111. Wilkinson, T. A.; Yin, J.; Pidgeon, C.; Post, C. B. Alkylation of cysteine-containing peptides to mimic palmitoylation J. Peptide Res. 55, 140-147.
112. Sulfur Ylides, Trost, B. M.; Melvin, S. M., 1975, Academic Press, New York, pp 344.
113. Vedejs, E. Sulfur-Mediated Ring Expansions in Total Synthesis Acc. Chem. Res. 1984, 17, 358-364.
114. Aggarwal, V.; Richardson, J. In Science of Synthesis: Compounds with Two Carbon-Heteroatom Bonds, Eds. Padwa, A., 2004, Thieme, Stuttgart, 21-104.
115. Kirmse, W.; Kapps, M. Reaktionen des diazomethans mit diallylsulfid and allyläthern unter kupfersalz-katalyse Chem. Ber. 1968, 101, 994-1003.
116. Doyle, M. P.; Tamblyn, W. H.; Bagheri, V. Highly Effective Catalytic Methods for Ylide Generation from Diazo Compounds. Mechanism of the Rhodium- and Copper-Catalyzed Reactions with Allylic Compounds J. Org. Chem. 1981, 46, 5094-5102.
117. Doyle, M. P.; Tamblyn, W. H.; Bagheri, V. Highly Effective Catalytic Methods for Ylide Generation from Diazo Compounds. Mechanism of the Rhodium- and Copper-Catalyzed Reactions with Allylic Compounds J. Org. Chem. 1981, 46, 4094-5102.
118. Doyle, M. P.; Griffin, J. H.; Chinn, M. S.; Van Leusen, D. Rearrangements of Ylides Generated from Reactions of Diazo Compounds with Allyl Acetals and Thioketals by Catalytic Methods. Heteroatom Acceleration of the [2,3]-Sigmatropic Rearrangement J. Org. Chem. 1984, 49, 1917-1925.
119. Ando, W.; Yagihara, T.; Kondo, S.; Nakayama, K.; Yamato, H.; Nakaido, S.; Migita, T. Reaction of carbethoxycarbene with aliphatic sulfides and allyl compounds J. Org. Chem. 1971, 36, 1732-1736.
120. Modern Catalytic Methods for Organic Synthesis with Diazo compounds, Doyle, M. P.; McKervey, M. A.; Ye, T., 1998, Wiley, New York.
121. Doyle, M. P.; Forbes, D. C. Recent Advances in Asymmetric Catalytic Metal Carbene Transformations Chem. Rev. 1998, 98, 911-936.
122. Hodgson, D. M.; Pierard, F. Y. T. M.; Stupple, P. A. Catalytic enantioselective rearrangements and cycloadditions involving ylides from diazo compounds Chem. Soc. Rev. 2001, 30, 50-61.
123. Li, A.-H.; Dai, L.-X.; Aggarwal, V. K. Asymmetric Ylide Reactions: Epoxidation, Cyclopropanation, Aziridination, Olefination, and Rearrangement Chem. Rev. 1997, 97, 2341-2372.
124. Mcfarland, J. M.; Francis, M. B. Reductive Alkylation of Proteins using Iridium Catalyzed Transfer Hydrogenation J. Am. Chem. Soc. 2005, 127, 13490-13491.
125. Van Maarseveen, J. H.; Reek, J. N. H.; Back, J. W. Transition-Metal Catalysis as a Tool for the Covalent Labeling of Proteins Angew. Chem. Int. Ed. 2006, 45, 1841-1843.
126. Antos, J. M.; Francis, M. F. Selective Tryptophan Modification with Rhodium Carbenoids in Aqueous Solution J. Am. Chem. Soc. 2004, 126, 10256-10257.
127. Nyong, A. M.; Rainier, J. D. The Diastereoselective Synthesis of Quaternary Substituted Thioindolines from Sulfur Ylide Intermediates J. Org. Chem. 2005, 70, 746-748.
128. Easton, C. J. Free-Radical Reactions in the Synthesis of a-Amino Acids and Derivatives Chem. Rev. 1997, 97, 53-82.
129. Easton, C. J. In Radicals in Organic Synthesis, Eds. Renaud, P.; Sibi, M. P., 2001, Wiley-VCH, Weinheim, 505-522.
130. Easton, C. J.; Hutton, C. A.; Roselt, P. D.; Tiekink, E. R. T. Stereocontrolled synthesis of b-hydroxyphenylalanine and b-hydroxytyrosine derivatives Tetrahedron 1994, 50, 7327-7340.
131. Easton, C. J.; Hutton, C. A.; Tan, E. W.; Tiekink, E. R. T. Synthesis of homochiral hydroxy-α-amino acid derivatives Tetrahedron Lett. 1990, 48, 7059-7062.
132. Mohapatra, D. K.; Durugkar, K. Efficient and selective cleavage of the tert-butoxycarbonyl (Boc) group under basic conditions ARKIVOC 2005, 20-28.
133. Kokotos, G.; Padron, J. M.; Martin, T.; Gibbons, W. A.; Martin, V. S. A General Approach to the Asymmetric Synthesis of Unsaturated Lipidic a-Amino Acids. The First Synthesis of a-Aminoarachidonic Acid J. Org. Chem. 1998, 63, 3741-3744.
134. Furukawa, N.; Morishita, T.; Akasaka, T.; Oae, S. A new acid-catalyzed rearrangement of thiosulfinates to a-acetylthiosulfoxides in acetic anhydride J. Chem. Soc., Perkin Trans. 2 1980, 432-440.
135. Fmoc Solid Phase Peptide Synthesis: A Practical Approach, Eds. Chan, W. C.; White, P. D., 2000, OUP, Oxford, pp 346.
136. Backes, B. J.; Virgilio, A. A.; Ellman, J. A. Activation Method to Prepare a Highly Reactive Acylsulfonamide “Safety-Catch” Linker for Solid-Phase Synthesis J. Am. Chem. Soc. 1996, 118, 3055-3056.
137. Backes, B. J.; Ellman, J. A. An Alkanesulfonamide “Safety-Catch” Linker for Solid-Phase Synthesis J. Org. Chem. 1999, 64, 2322-2330.
138. Wittinghofer, A.; Waldmann, H. RAS-A molecular switch involved in tumor formation Angew. Chem. Int. Ed. 2000, 39, 4192-4214.
139. Park, H.-W.; Boduluri, S. R.; Moomaw, J. F.; Casey, P. J.; Beese, L. S. Crystal structure of protein farnesyltransferase at 2.25 angstrom resolution Science 1997, 275, 1800-1804.
140. Robins, M. J.; Wilson, J. S.; Hansske, F. A general procedure for the efficient deoxygenation of secondary alcohols. Regiospecific and stereoselective conversion of ribonucleosides to 2′-deoxynucleosides J. Am. Chem. Soc. 1983, 105, 4059-4065.
141. Crich, D.; Li, H. Direct Stereoselective Synthesis of b-Thiomannopyranosides J. Org. Chem. 2000, 56, 801-805.
142. Crich, D.; De La Mora, M. A.; Cruz, R. Synthesis of the Mannosyl Erythritol Lipid MEL A; Confirmation of the Configuration of the “meso-erythritol” Moiety Tetrahedron 2002, 58, 35-44.
143. Barresi, F.; Hindsgaul, O. Chemically Synthesized Oligosaccharides, 1994. A Searchable Table of Glycosidic Linkages J. Carbohydr. Chem. 1995, 14, 1043-1087.
144. Schmidt, R. R.; Jung, K.-H. In Carbohydrates in Chemistry and Biology, Eds. Ernst, B.; Hart, G. W.; Sinaÿ, P., 2000, Wiley-VCH, Weinheim, 5-59.
145. Chemistry of Saccharides: Chemical Synthesis of Glycosides and Glycomimetics, Eds. Ernst, B.; Hart, G. W.; Sinaÿ, P., 2000, Wiley-VCH, Weinheim, pp 585.
146. Glycoscience: Chemistry and Chemical Biology, Eds. Fraser-Reid, F.; Kuniaki, T.; Thiem, J., 2001, Springer-Verlag, Berlin, vols 1-3.
147. Bioorganic Chemistry: Carbohydrates, Eds. Hecht, S. M., 1999, OUP, New York, pp 624.
148. Carbohydrates: Structure and Biology, Lehmann, J., 1998, Thieme, Stuttgart, pp 274.
149. Bertozzi, C. R.; Kiessling, L. L. Chemical Glycobiology Science 2001, 291, 2357-2364.
150. Boucaut, J.-C.; Darribère, T.; Poole, T. J.; Aoyama, H.; Yamada, K. M.; Thiery, J. P. Biologically Active Synthetic Peptides as Probes of Embryonic Development: A Competitive Peptide Inhibitor of Fibronectin Function Inhibits Gastrulation in Amphibian Embryos and Neural Crest Cell Migration in Avian Embryos J. Cell. Biol. 1984, 99, 1822-1830.
151. Yamada, K. M.; Kennedy, D. W. Dualistic Nature of Adhesive Protein Function: Fibronectin and Its Biologically Active Peptide Fragments Can Autoinhibit Fibronectin Function J. Cell. Biol. 1984, 99, 29-36.
152. Ferrier, R. J.; Zubkov, O. A. Transformation of glycals into 2,3-unsaturated glycosyl derivatives Org. React. 2003, 62, 569-736.
153. Fraser-Reid, B.; Radatus, B. 4,6-Di-O-acetyl-aldehydro-2,3-dideoxy-D-erythro-trans-hex-2-enone. A probable reason for the “al” in Emil Fischer's triacetyl glucal J. Am. Chem. Soc. 1970, 92, 5288-5290.
154. The Anomeric Effect, Juaristi, E.; Cuevas, G., 1995, CRC, Boca Raton, pp 229.
155. Tvaroska, I.; Bleha, T. Anomeric and Exoanomeric Effects in Carbohydrate Chemistry Adv. Carbohydr. Chem. Biochem. 1989, 47, 45-123.
156. Crich, D. In Radicals in Organic Synthesis, Eds. Renaud, P.; Sibi, M., 2001, Wiley-VCH, Weinheim, 188-206.
157. Giese, B.; Gilges, S.; Groninger, K. S.; Lamberth, C.; Witzel, T. acyloxy shift, carbohydrate examples Liebigs 1988, 615.
158. Giese, B.; Groninger, K. S. acyloxy shift, 2-deoxy glycoside Org. Synth. 1990, 69, 66.
159. Kim, H. J.; Men, H.; Lee, C. Stereoselective Palladium-Catalyzed O-Glycosylation using glycals J. Am. Chem. Soc. 2004, 126, 1336-1337.
160. Babu, R. S.; Zhou, M.; O'doherty, G. A. De Novo Synthesis of Oligosaccharides using a palladium-catalyzed glycosylation reaction J. Am. Chem. Soc. 2004, 126, 3428-3429.
161. Comely, A. C.; Eelkema, R.; Minnaard, A. J.; Fering a, B. L. De Novo Asymmetric Bio- and Chemocatalytic Synthesis of Saccharides—Steroselective Formal O-Glycoside Bond Formation Using Palladium Catalysis J. Am. Chem. Soc. 2003, 125, 8714-8715.
162. Taillefumier, C.; Chapleur, Y. Synthesis and Uses of exo-Glycals Chem. Rev. 2004, 104, 263-292.
163. Volante, R. P. A new, highly efficient method for the conversion of alcohols to thiolesters and thiols Tetrahedron Lett. 1981, 22, 3119-3122.
164. Hughes, D. L. The Mitsunobu Reaction Org. React. 1992, 42, 335-656.
165. Driguez, H. In Topics in Current Chemistry: Glycoscience, Eds. Driguez, H.; Thiem, J., 1997, Springer, Berlin, 85-116.
166. Sulzenbacher, G.; Driguez, H.; Henrissat, B.; Schulein, M.; Davies, G. J. Structure of the Fusarium oxysporum Endoglucanae I with a Nonhydrolyzable Substrate Analogue: Substrate Distortion Gives Rise to the Preferred Axial Orientation for the Leaving Group Biochemistry 1996, 35, 15280-15287.
167. Bernardes, G. J. L.; Gamblin, D. P.; Davis, B. G. The Direct Formation of Glycosyl Thiols from Reducing Sugars Allows One-Pot Protein Glycoconjugation Angew. Chem. Int. Ed. 2006, 45, in press.
168. Johnson, C. R.; Johns, B. A. Suzuki Cross-coupling of Carbohydrates: Synthesis of b-Arylmethyl-C-glycosides and Aryl-Scaffolded Trisaccharide Mimics Synlett 1994, 1406-1408.
169. Kapur, M.; Khartulyari, A.; Maier, M. E. Stereselective synthesis of protected 1,2-diols and 1,2,3-triols by a tandem hydroboration-coupling sequence Org. Lett. 2006, 8, 1629-1632.
170. Veronese, F. M.; Pasut, G. Pegylation, successful approach to drug delivery Drug. Disc. Today 2005, 10, 1451-1458.
171. Harris, M. J.; Chess, R. B. Effect of Pegylation on Pharmaceuticals Nat. Rev. Drug. Disc. 2003, 2, 214-221.
172. Shaunak, S.; Godwin, A.; Choi, J.-W.; Balan, S.; Pedone, E.; Vijayarangam, D.; Heidelberger, S.; Teo, I.; Zloh, M.; Brocchini, S. Site-specific pegylation of native disulfide bonds in therapeutic proteins Nature Chem. Biol. 2006, 2, 312-313.
173. Doherty, D. H.; Rosendahl, M. S.; Smith, D. J.; Hughes, J. M.; Chlipala, E. A.; Cox, G. N. Site-Specific PEGylation of Engineered Cysteine Analogues of Recombinant Human Granulocyte-Macrophage Colony-Stimulating Factor Bioconj. Chem. 2005, 16, 1291-1298.
174. Manjula, B. N.; Tsai, A.; Upadhya, R.; Perumalsamy, K.; Smith, P. K.; Malavalli, A.; Vanderegriff, K.; Winslow, R. M.; Intaglietta, M.; Prabhakaran, M.; Friedman, J. M.; Acharya, A. S. Site-Specifc PEGylation of Hemoglobin at Cys-93(b): Correlation between the Colligative Properties of the PEGylated Protein and the Length of the Conjugated PEG Chain Bioconj. Chem. 2003, 14, 464-472.
175. Juszczak, L. J.; Manjula, B.; Bonaventura, C.; Acharya, S. A.; M., F. J. UV Resonance Raman Study of d93-Modified Hemoglobin A: Chemical Modifier-Specific Effects and Added Influences of Attached Poly(ethylene glycol) Chains Biochemistry 2002, 41, 376-385.
176. Zalipsky, S.; Qazen, M.; Walker, J. A.; Mullah, N.; Quinn, Y. P.; Huang, S. K. New Detachable Poly(ethylene glycol) Conjugates: Cysteine-Cleavable Lipopolymers Regenerating Natural Phospholipid, Diacyl Phosphatidylethanolamine Bioconj. Chem. 1999, 10, 703-710.
177. Toy, P. H., Janda, T. K. Soluble Polymer-Supported Organic Synthesis Acc. Chem. Res. 2000, 33, 546-554.
178. Gravert, D. J.; Janda, K. D. Organic Synthesis on Soluble Polymer supports: Liquid-Phase Methodologies Chem. Rev. 1997, 97, 489-509.
179. For the classical disulfide ligation as means of attachment of spin labels to proteins see: Mehboob, S.; Luo, B.-H.; Fu, W.; Johnson, M. E.; Fung, L. W.-M. Conformational Studies of the Tetramerization Site of Human Erythroid Spectrin by Cysteine-Scanning Spin-Labeling EPR Methods Biochemistry 2005, 44, 15898-15905.
180. Kohanski, R. A. In Encyclopedia of Biological Chemistry, Eds. Lennarz, W. J.; Lane, M. D., 2004, Elsevier, Oxford, 179-181.
181. Giepmans, B. N. G.; Adams, S. R.; Ellisman, M. H.; Tsien, R. Y. The Fluorescent Toolbox for Assessing Protein Location and Function Science 2006, 312, 217-224.
182. Waggoner, A. Fluorescent labels for proteomics and genomics Curr. Opin. Chem. Biol. 2006, 10, 62-66.
183. Daly, C. J.; Mcgrath, J. C. Fluorescent ligands, antibodies, and proteins for the study of receptors Pharmacol. Therapeutics 2003, 100, 101-118.
184. Brittain, S. M.; Ficarro, S. B.; Brock, A.; Peters, E. C. Enrichment and analysis of peptide subsets using fluorous affinity tags and mass spectrometry Nature Biotechnology 2005, 23, 463-468.
185. Hackeng, T. M.; Griffin, J. H.; Dawson, P. E. Protein synthesis by native chemical ligation: expanded scope by using straightforward methodology Proc. Natl. Acad. Sci., U.S.A. 1999, 96, 10068-10073.
186. Diazo compounds: properties and synthesis, Regitz, M.; Maas, G., 1986, Academic Press, Orlando, pp 596.
187. Seitz, O. In Carbohydrate-based Drug Discovery, Eds. Wong, C. H., 2003, Wiley-VCH, Weinheim, 169-214.
188. For a succinct discussion of these problems see: Cohen-Anisfeld, S. T.; Lansbury, P. T. A Practical, Convergent Method for Glycopeptide Synthesis J. Am. Chem. Soc. 1993, 115, 10531-10537.
189. Tanaka, H.; Iwata, Y.; Takahashi, D.; Adachi, M.; Takahashi, T. Efficient Stereoselective Synthesis of g-N-Glycosyl Asparagines by N-Glycosylation of Primary Amide Groups J. Am. Chem. Soc. 2005, 127, 1630-1631.
190. Wang, Z., -G.; Warren, J. D.; Dudkin, V. Y.; Zhang, X.; Iserloh, U.; Visser, M.; Eckhardt, M.; Seeberger, P. H.; Danishefsky, S. J. A highly convergent synthesis of an N-linked glycopeptide presenting the H-type 2 human blood group determinant Tetrahedron 2006, 62, 4954-4978.
191. Kan, T.; Fukuyama, T. Ns strategies: a highly versatile synthetic method for amines Chem. Commun. 2004, 353-359.
192. Messeri, T.; Sternbach, D. D.; Tomkinson, N. C. O. A Novel Deprotection/Functionalization Sequence using 2,4-Dinitrobenzenesulfonamide: Part 1 Tetrahedron Lett. 1998, 39, 1669-1672.
193. Messeri, T.; Sternbach, D. D.; Tomkinson, N. C. O. A Novel Deprotection/Functionalization Sequence using 2,4-Dinitrobenzenesulfonamide: Part 2 Tetrahedron Lett. 1998, 39, 1673-1676.
194. Turner, J. J.; Wilschut, N.; Overkleeft, H. S.; Klaffke, W.; Van Der Marel, G.; Van Boom, J. H. Mitsunobu glycosylation of nitrobenzenesulfonamides: novel route to Amadori rearrangement products Tetrahedron Lett. 1999, 40, 7039-7042.
195. Huo, C.; Wang, C.; Zhao, M.; Peng, S. Stereoselective synthesis of natural N-(1-deoxy-D-b-fructos-1-yl)-L-amino acids and their effectiveness on lead decomposition Chem. Res. Toxicol. 2004, 17, 1112-1120.
196. Ragupathi, G.; Slovin, S. F.; Adluri, S.; Sames, D.; Kim, I. J.; Kim, H. M.; Spassova, M.; Bornmann, W. G.; Lloyd, K. O.; Scher, H. I.; Livingston, P. O.; Danishefsky, S. J. A Fully Synthetic Globo H Carbohydrate Vaccine Induces a Focused Humoral Response in Prostate Cancer Patients: a Proof of Principle Angew. Chem. Int. Ed. 1999, 38, 563-566.
197. Spassova, M. K.; Bornmann, W. G.; Ragupathi, G.; Sukenick, G.; Livingston, P. O.; Danishefsky, S. J. Synthesis of selected LeY and KH-1 analogues: a medicinal chemistry approach to vaccine optimization J. Org. Chem. 2005, 70, 3383-3395.
198. Mukaiyama, T. New synthetic reactions based on the onium salts of aza-arenes Angew. Chem. Int. Ed. 1979, 18, 707-808.
199. Blakemore, P. R.; Cole, W. J.; Kocienski, P. J.; Morley, A. A Stereoselective Synthesis of trans-1,2, Disubstituted Alkenes Based on the Condensation of Aldehydes with Metallated 1-Phenyl-1H-tetrazol-5-yl Sulfones Synlett 1998, 26-28.
200. Alonso, D. A.; Fuensanta, M.; Nájera, C.; Varea, M. 3,5-Bis(trifluoromethyl)phenyl Sulfones in the Direct Julia-Kocienski Olefination J. Org. Chem. 2005, 70, 6404-6416.
201. Camarero, J. A.; Hackel, B. J.; De Yoreo, J. J.; Mitchell, A. R. Fmoc-Based Synthesis of Peptide a-Thioesters Using an Aryl Hydrazine Support J. Org. Chem. 2004, 69, 4145-4151.
202. Ingenito, R.; Bianchi, E.; Fattori, D.; Pessi, A. Solid Phase Synthesis of Peptide C-Terminal Thioesters by Fmoc/t-Bu Chemistry J. Am. Chem. Soc. 1999, 121, 11369-11364.
203. Shin, Y.; Winans, K. A.; Backes, B. J.; Kent, S. B. H.; Ellman, J. A.; Bertozzi, C. R. Fmoc-Based Synthesis of Peptide-a-Thioesters: Application to the Total Chemical Synthesis of a Glycoprotein by Native Chemical Ligation J. Am. Chem. Soc. 1999, 121, 11684-11689.
204. Brask, J.; Albericio, F.; Jensen, K. J. Fmoc Solid-phase Synthesis of Peptide Thioesters by Masking as Trithioortho Esters Org. Lett. 2003, 5, 2951-2953.
205. Swinnen, D.; Hilvert, D. Facile, Fmoc-compatible solid-phase synthesis of peptide C-terminal thioesters 2000, 2, 2439-2442.
206. Kenner, G. W.; Mcdermott, J. R.; Sheppard, R. C. The Safety Catch Principle in Solid Phase Peptide Synthesis J. Chem. Soc., Chem. Commun. 1971, 636-637.
207. Shao, N.; Xue, J.; Guo, Z. W. Chemical synthesis of a skeleton structure of sperm CD52—a GPI-anchored glycopeptide Angew. Chem. Int. Ed. 2004, 43, 1569-1573.
208. Crich, D.; Banerjee, A. Stereocontrolled Synthesis of the D- and L-glycero-b-D-mannoheptopyranosides and their 6-Deoxy Analogs. Synthesis of Methyl α-L-Rhamnopyranosyl-(1→3)-D-glycero-b-D-manno-heptopyranosyl-(1→3)-6-deoxy-glycero-b-D-manno-heptopyranosyl-(1→4)-a-L-rhamnopyranoside, a Tetrasaccharide Subunit of the Lipopolysaccharide from Plesimonas shigelloides J. Am. Chem. Soc. 2006, 128, in press (asap).
209. Crich, D.; Banerjee, A.; Yao, Q. Direct Chemical Synthesis of the β-D-Mannans: the β-(1,2)- and β-(1,4)-Series J. Am. Chem. Soc. 2004, 126, 14930-14934.
210. Protective Groups in Organic Synthesis, Greene, T. W.; Wuts, P. G. M., 1999, Wiley, New York, pp 779.
211. Protecting Groups, Kocienski, P. J., 2005, Thieme, Stuttgart, pp 679.
212. Chemical Approaches to the Synthesis of Peptides and Proteins, Lloyd-Williams, P.; Albericio, F.; Giralt, E., 1997, CRC, Boca Raton, pp 297.
213. Houben-Weyl. Methods in Organic Chemistry. Synthesis of Peptides and Peptidomimetics, Eds. Goodman, M.; Mahwah, A. F.; Moroder, L.; Toniolo, C., 2002-2003, Thieme, Stuttgart.
214. Peptide Chemistry. A Practical Textbook, Bodanszky, M., 1993, Springer, Berlin, pp 198.
215. Principles of Peptide Synthesis, Bodanszky, M., 1984, Springer Verlag, Berlin, pp 307.
216. The Practice of Peptide Synthesis, Bodanszky, M.; Bodanszky, A., 1994, Springer-Verlag, Heidelberg, pp.
217. Barton, D. H. R.; Chavis, C.; Kaloustian, M. K.; Magnus, P. D.; Poulton, G. A.; West, P. J. Photochemical transformations. XaaI. Photolysis of thiobenzoic acid O-esters. II. General methods for the preparation of thiobenzoic acid O-esters. J. Chem. Soc. Perkin Trans. 11973, 1571-1574.
218. Somlai, C.; Nyerges, L.; Penke, B.; Hegyes, P.; Voelter, W. Design, synthesis and potential use of 3,9-substituted xanthene handles for solid phase peptide synthesis. J. Prakt. Chem./Chem. Zeit. 1994, 336, 429-433.
219. Capuano, L.; Zander, R.; Bolourtschi, A. Nucleophilic substitution by exchange of the carbamoyloxy group. III. Synthesis of 9-xanthenyl- and 9-thioxanthenyl esters, ethers, and amines Chem. Ber. 1971, 104, 3750-3756.
220. Kayed, R.; Head, E.; Thompson, J. L.; Mcintire, T. M.; Milton, S. C.; Cotman, C. W.; Glabe, C. G. Common Structure of Soluble Amyloid Oligomers Implies Common Mechanism of Pathogenesis Science 2003, 300, 486-489.
221. Dahlgren, K. N.; Manelli, A. M.; Stine, W. B.; Baker, L. K.; Krafft, G. A.; Ladu, M. J. Oligomeric and Fibrillar Species of Amyloid-Peptides Differentially Affect Neuronal Viability J. Biol. Chem. 2002, 277, 32046-32053.
222. Roher, A. E.; Chaney, M. 0.; Kuo, Y.-M.; Webster, S. D.; Stine, W. B.; Haverkamp, L. J.; Woods, A. S.; Cotter, R. J.; Tuohy, J. M.; Krafft, G. A.; Bonnell, B. S.; Emmerling, M. R. Morphology and Toxicity of A-(1-42) Dimer Derived from Neuritic and Vascular Amyloid Deposits of Alzheimer's Disease J. Biol. Chem. 2002, 271, 20631-20635.
223. Kuo, Y.-M.; Emmerling, M. R.; Vigo-Pelfrey, C.; Kasunic, T. C.; Kirkpatrick, J. B.; Murdoch, G. H.; Ball, M. J.; Roher, A. E. Water-soluble A(N-40, N-42) Oligomers in Normal and Alzheimer Disease Brains J. Biol. Chem. 2002, 271, 4077-4081.
224. Curtain, C. C.; Ali, F.; Volitakis, I.; Chemy, R. A.; Norton, R. S.; Beyreuther, K.; Barrow, C. J.; Masters, C. L.; Bush, A. I.; Barnham, K. J. Alzheimer's Disease Amyloid-Binds Copper and Zinc to Generate an Allosterically Ordered Membrane-penetrating Structure Containing Superoxide Dismutase-like Subunits J. Biol. Chem. 2001, 271, 20466-20473.
225. Fukuda, H.; Shimizu, T.; Nakajima, M.; Mori, H.; Shirasawa, T. Synthesis, aggregation, and neurotoxicity of the alzheimer's Aβ1-42 amyloid peptide and its isoaspartyl isomers 1999, 9, 953-956.
226. Dong, J.; Atwood, C. S.; Anderson, V. E.; Siedlak, S. L.; Smith, M. A.; Perry, G.; Carey, P. R. Metal Binding and Oxidation of Amyloid—within Isolated Senile Plaque Cores Raman Microscopic Evidence Biochemistry 2003, 42, 2768-2773.
227. Schoneich, C.; Pogocki, D.; Hug, G. L.; Bobrowski, K. Free Radical Reactions of Methionine in Peptides: Mechanisms Relevant to b-Amyloid Oxidation and Alzheimer's Disease J. Am. Chem. Soc. 2003, 125, 13700-13713.
228. Sohma, Y.; Hayashi, Y.; Kimura, M.; Chiyomori, Y.; Taniguchi, A.; Sasaki, M.; Kimura, T.; Kiso, Y. The O-acyl isopeptide method for the synthesis of difficult sequence-containing peptides: application to the synthesis of Alzheimer's disease-related amyloid peptide (A) 1-42 J. Pept. Sci. 2005, 11, 441-451.
229. Kim, Y. S.; Moss, J. A.; Janda, K. D. Biological Tuning of Synthetic Tactics in Solid-Phase Synthesis: Application to Ab(1-42) J. Org. Chem. 2004, 69, 7776-7778.
230. Krishnakumar, I. M.; Mathew, B. Polyethyleneglycol graft copoly(styrene-1,4-butanediol dimethacrylate) resin for gel-phase peptide synthesis Lett. Pept. Sci. 2002, 8, 339-347.
231. Tickler, A. K.; Barrow, C. J.; Wade, J. D. Improved preparation of amyloid-b-peptides using DBU as N-a-Fmoc deprotection reagent J. Pept. Sci. 2001, 7, 488-494.
232. Clippindale, A. B.; Macris, M.; Wade, J. D.; Barrow, C. J. Synthesis and secondary structural studies of penta(acetyl-Hmb)Ab(1±40) J. Pept. Res. 1999, 53, 665-672.
233. García-Martín, F.; Quintanar-Audelo, M.; García-Ramos, Y.; Cruz, L. J.; Gravel, C.; Furic, R.; Côté, S.; Tulla-Puche, J.; Albericio, F. ChemMatrix, a Poly(ethylene glycol)-Based Support for the Solid-Phase Synthesis of Complex Peptides J. Comb. Chem. 2006, 8, 213-220.
234. Inui, T.; Bódi, J.; Nishio, H.; Nishiuchi, Y.; Kimura, T. Synthesis of amyloid b-peptides in solution employing chloroform-phenol mixed solvent for facile segment condensation of sparingly soluble protected peptides Lett. Pept. Sci. 2002, 8, 319-330.
235. Villain, M.; Vizzavona, J.; Gaertner, H. Chemical ligation of multiple peptide fragments using a new protection strategy Proc. Second Int. Seventeenth Am. Peptide Symp. 2001, 107-108.
236. Villain, M.; Vizzavona, J.; Rose, K. Covalent capture: a new tool for the purification of synthetic and recombinant polypeptides Chem. Biol. 2001, 8, 673-679.
237. Schnolzer, M.; Alewood, P.; Jones, A.; Alewood, D.; Kent, S. B. H. In situ neutralization in Boc-chemistry solid phase peptide synthesis. Rapid, high yield assembly of difficult sequences Int. J. Peptide Protein Res. 1992, 40, 180-193.
238. Crich, D. B., F.; Krishnamurthy, V. “Allylic Disulfide Rearrangement and Desulfurization: Mild, Electrophile-Free Thioether Formation From Thiols,” Org. Lett. 2006, 8, 3593-3596.

1. A method for ligation or derivatization of a peptide, which comprises reacting sulfonamide (I) with peptide (II) to form ligated peptide (III): 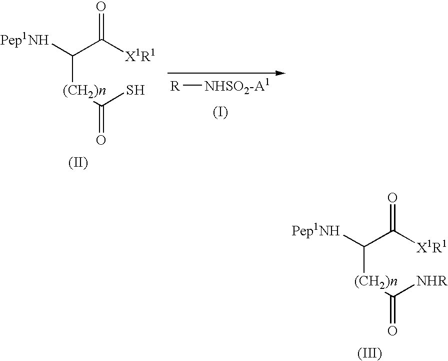
wherein R is an amino acid, a peptide, a monosaccharide, or a polysaccharide, optionally including one or more protecting groups on a nitrogen, oxygen, or sulfur substitutent thereof; X¹ is O or NH; R¹ is an alkyl group, an alkenyl group, an aryl group, an alkyl-substituted aryl group, an aryl-substituted alkyl group, an amino acid, or a peptide, optionally including one or more protecting groups on a nitrogen, oxygen, or sulfur substitutent thereof; A¹ is an electron deficient alkyl, aryl, or heteroaryl group; Pep¹ is an amino acid or a peptide, which optionally includes one or more protecting groups on a nitrogen, oxygen, or sulfur substitutent thereof; and n is 1 or 2.

2. The method of claim 1 wherein X¹ is NH; and R¹ is an amino acid or a peptide, optionally comprising one or more protecting groups on a nitrogen, oxygen, or sulfur substitutent thereof, or an amino acid.

3. The method of claim 1 wherein X¹ is NH; and R¹ is a benzyl group or a C₁-C₄ alkyl group.

4. The method of claim 1 wherein R is a an amino acid or a peptide, optionally comprising one or more protecting groups on a nitrogen, oxygen, or sulfur substitutent thereof.

5. The method of claim 1 wherein R is a polysaccharide or a polysaccharide comprising one or more protecting groups on a nitrogen, oxygen, or sulfur substitutent thereof.

6. The method of claim 1 wherein A¹ is a nitro-substituted aryl group, a fluoroalkyl-substituted aryl group, an aryl tetrazole, or a pyridium group.

7. The method claim 1 wherein A¹ is a nitro-substituted aryl group.

8. The method of claim 7 wherein the a nitro-substituted aryl group is a nitro-substituted phenyl group.

9. A method for glycosylation of a peptide, which comprises reacting a N-glycosyl-o-nitrobenzylamino-disulfide compound (Glyc¹-NHZ) with peptidyl thioester (IV) in the presence of a thiol, and subsequent photolysis to afford glycosylated peptide (V): 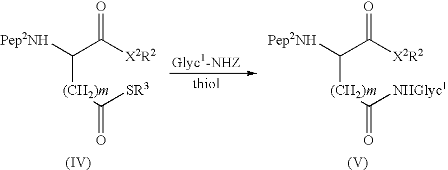
wherein X² is O or NH; R² is an alkyl group, an alkenyl group, an aryl group, an alkyl-substituted aryl group, an aryl-substituted alkyl group, an amino acid, or a peptide, optionally including one or more protecting groups on a nitrogen, oxygen, or sulfur substitutent thereof; R³ is an alkyl group, an alkenyl group, an aryl group, an alkyl-substituted aryl group, or an aryl-substituted alkyl group; Pep² is an amino acid or a peptide, which optionally includes one or more protecting groups on a nitrogen, oxygen, or sulfur substitutent thereof; Glyc¹ is a monosaccharide or a polysaccharide, optionally including one or more protecting groups on a nitrogen, oxygen, or sulfur substitutent thereof; Z is an o-nitrobenzyl-disulfide moiety; and m is 1 or 2.

10. The method of claim 9 wherein R³ is C₁-C₄ alkyl or benzyl.

11. The method of claim 9 wherein R³ is ethyl.

12. The method of claim 9 wherein the thiol is a 2-mercaptoethanesulfonic acid salt.

13. The method of claim 9 wherein Glyc¹-NHZ has the following structure: 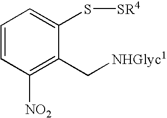
wherein R⁴ is an alkyl group, an alkenyl group, an aryl group, an alkyl-substituted aryl group, or an aryl-substituted alkyl group.

14. The method of claim 10 wherein Glyc¹-NHZ has the following structure: 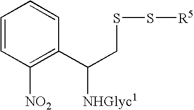
wherein R⁵ is an alkyl group, an alkenyl group, an aryl group, an alkyl-substituted aryl group, or an aryl-substituted alkyl group.

15. The method of claim 9 wherein X² is NH; and R² is a peptide or an amino acid, optionally comprising one or more protecting groups on a nitrogen, oxygen, or sulfur substitutent thereof.

16. A method for native chemical ligation of a peptide, which comprises reacting peptidyl thioester (IV) with aminodisulfide (VI) in the presence of a thiol to afford mercapto peptide (VII); and optionally reducing (VII) to afford peptide (VIII): 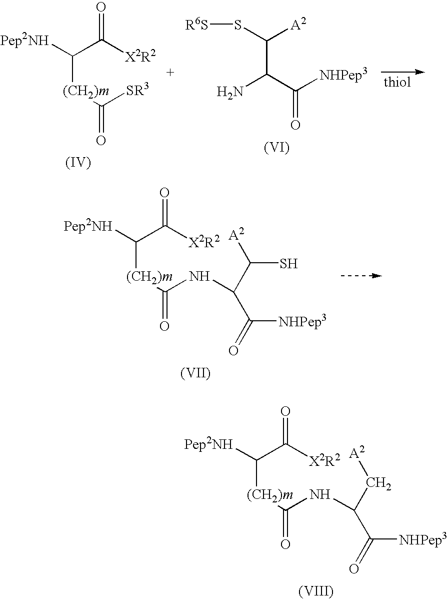
wherein X² is O or NH; R² is an alkyl group, an alkenyl group, an aryl group, an alkyl-substituted aryl group, an aryl-substituted alkyl group, an amino acid, or a peptide, optionally including one or more protecting groups on a nitrogen, oxygen, or sulfur substitutent thereof; R³ and R⁶ are each independently an alkyl group, an alkenyl group, an aryl group, an alkyl-substituted aryl group, or an aryl-substituted alkyl group; Pep² and Pep³ are each independently an amino acid or a peptide, which optionally includes one or more protecting groups on a nitrogen, oxygen, or sulfur substitutent thereof; A² is phenyl, 4-hydroxyphenyl, or 3-indolyl; and m is 1 or 2.

17. The method of claim 16 wherein R³ and R⁶ are each independently C₁-C₄ alkyl or benzyl.

18. The method of claim 16 wherein R³ and R⁶ are each ethyl.

19. The method of claim 16 wherein the thiol is a 2-mercaptoethanesulfonic acid salt.

20. The method of claim 16 wherein X² is NH; and R² is a peptide or an amino acid, optionally comprising one or more protecting groups on a nitrogen, oxygen, or sulfur substitutent thereof.
